(19) United States
(12) Patent Application Publication
(10) Pub. No.: US 2024/0026381 A1
(43) Pub. Date: Jan. 25, 2024

(54) SPLIT PRIME EDITING PLATFORMS

(71) Applicant: The Board of Trustees of the University of Illinois, Urbana, IL (US)

(72) Inventors: Pablo Perez-Pinera, Mahomet, IL (US); Wendy Woods, Champaign, IL (US); Jackson Winter, Urbana, IL (US); Michael Gapinske, Urbana, IL (US)

(73) Assignee: The Board of Trustees of the University of Illinois, Urbana, IL (US)

(21) Appl. No.: 18/251,505

(22) PCT Filed: Nov. 3, 2021
(86) PCT No.: PCT/US2021/057908

§ 371 Date: May 2, 2023

Related U.S. Application Data
(60) Provisional application No. 63/109,131, filed on Nov. 3, 2020.

(51) Int. Cl. C12N 15/90; C12N 15/11; C12N 9/22; C12N 9/12

(52) U.S. Cl. CPC C12N 15/90; C12N 15/11; C12N 9/22; C12N 9/1276; C12Y 207/07049; C12N 2310/20

(57) ABSTRACT

The present disclosure provides prime editors, editor systems and methods of uses thereof. Specifically, the disclosure provides methods of use of split prime editors for editing genomic DNA.

### PRIORITY

This application claims the benefit of U.S. Ser. No. 63/109,131, filed on Nov. 3, 2020, which is incorporated by reference in its entirety.

### GOVERNMENT FUNDING

This invention was made with government support under Grant Number 1R01GM12749 awarded the National Institutes of Health. The government has certain rights in the invention.

### FIELD OF THE INVENTION

The present disclosure relates to split prime editors and to systems using split prime editors for editing genomic DNA.

### BACKGROUND

The implementation of prime editors for in vivo gene correction is limited by the fact that their size exceeds the carrying capacity of a single AAV vector. Therefore, there is a need for prime editing systems that overcome the packaging limitations associated with delivering prime editors and that can be used for research purposes or other in vivo applications such as treating human diseases.

### SUMMARY

Provided herein are split prime editors comprising a Cas nickase and an engineered reverse transcriptase, systems for prime editing, and methods of editing genomic DNA in a cell.

An embodiment provides a prime editor comprising: a first polynucleotide molecule encoding a Cas protein and an N-terminal fragment of a dimerization protein; and a second polynucleotide molecule encoding a C-terminal fragment of and a reverse transcriptase.

The N-terminal fragment of a dimerization protein can be the N-terminal fragment of an intein. The C-terminal fragment of a dimerization protein can be the C-terminal fragment of an intein. The first polynucleotide molecule and the second polynucleotide molecule can each comprise a promoter. The Cas protein can be a Cas nickase, a Cas9 nickase, a dead Cas protein, a dead Cas9, or an active Cas protein. The Cas9 nickase can be a Cas9 protein having an amino acid substitution at position 10 or at position 840 or at position 863. The Cas9 nickase can be D10A Cas9, D10N Cas9, H840N Cas9, H840Y Cas9, H840A Cas9, or N863A Cas9. The C-terminal fragment of a dimerization protein and the N-terminal fragment of a dimerization protein can be derived from PhoRadA, RmaDnaB^{Δ286}, SspDnaB^{Δ275}, SspDnaB^{M86Δ275}, SspDnaX, TvoVMA, NpuDnaE, NpuDnaB^{Δ283}, SspGyrB, AceL-TerL, PchPRP8, PfuRIR1-1, Psp-GDBPol-1, MtuRecA^{Δ228}, PfuRIR1-2, SceVMA^{Δ206}, RmaDnaB^{Δ271}, MtuRecA^{Δ285}, SspDnaB^{Δ274}, gp41-8, SceVMA^{Δ227}, IMPDH-1, NrdJ-1, MtuRecA^{Δ297} gp41-1, AovDnaE, AspDnaE, AvaDnaE, Cra(C5505)DnaE, Csp(CCY0110)DnaE, Csp(PCC8801)DnaE, CwaDnaE, Maer(NIES843)DnaE, Mcht(PCC7420)DnaE, MtuRecA^{Δ300}, NspDnaE, OliDnaE, Sel(PC7942)DnaE, SspDnaE, Ssp(PCC7002)DnaE, TerDnaE-3, TelDnaE, TvuDnaE, NeqPol, TerThyX^{Δ132}, or combinations thereof. An amino acid sequence of the N-terminal fragment of an intein can comprise SEQ ID NO:3. An amino acid sequence of the C-terminal fragment of an intein can comprise SEQ ID NO:4. The reverse transcriptase can be an M-MLV reverse transcriptase, a Marathon reverse transcriptase, a Rous sarcoma virus reverse transcriptase, an HIV-1 reverse transcriptase, an AMV reverse transcriptase, a telomerase reverse transcriptase, or any variant thereof. The first polynucleotide molecule can comprise one or more nuclear localization signals and the second polynucleotide molecule can comprise one or more nuclear localization signals. The first polynucleotide can encode a polypeptide molecule comprising SEQ ID NO:1. The second polynucleotide molecule can further comprise a linker. The linker can encode a polypeptide molecule comprising SEQ ID NO:22, 23, 24, 25, or 26. The first polynucleotide molecule can comprise one or more polynucleotides encoding a protein tag and the second polynucleotide molecule can comprise one or more polynucleotides encoding a protein tag.

Another embodiment provides a system for prime editing comprising: a first vector comprising a first polynucleotide molecule encoding a Cas protein and an N-terminal fragment of a dimerization protein; and a second vector comprising a second polynucleotide molecule encoding a C-terminal fragment of a dimerization protein and a reverse transcriptase. The Cas protein can be a Cas nickase, a Cas9 nickase, a dead Cas protein, a dead Cas9, or an active Cas protein.

An embodiment provides a prime editor comprising: a first polynucleotide molecule encoding a reverse transcriptase and an N-terminal fragment of a dimerization protein; and a second polynucleotide molecule encoding a C-terminal fragment of a dimerization protein and a Cas protein.

The N-terminal fragment of a dimerization protein can be the N-terminal fragment of an intein. The C-terminal fragment of a dimerization protein can be the C-terminal fragment of an intein. The Cas protein can be a Cas nickase, a Cas9 nickase, a dead Cas protein, a dead Cas9, or an active Cas protein. The Cas9 nickase can be a Cas9 protein having an amino acid substitution at position 10, at position 840, or at position 863. The Cas9 nickase can be a D10A Cas9, D10N Cas9, H840N Cas9, H840Y Cas9, H840A Cas9, or N863A Cas9. The first polynucleotide molecule and the second polynucleotide molecule can each comprise a promoter. The C-terminal fragment of a dimerization protein and the N-terminal fragment of a dimerization protein can be derived from PhoRadA, RmaDnaBΔ286, SspDnaBΔ275, SspDnaBM86Δ275, SspDnaX, TvoVMA, NpuDnaE, NpuDnaBΔ283, SspGyrB, AceL-TerL, PchPRP8, PfuRIR1-1, Psp-GDBPol-1, MtuRecAΔ228, PfuRIR1-2, SceVMAΔ206, RmaDnaBΔ271, MtuRecAΔ285, SspDnaBΔ274, gp41-8, SceVMAΔ227, IMPDH-1, NrdJ-1, MtuRecAΔ297, gp41-1, AovDnaE, AspDnaE, AvaDnaE, Cra(C5505)DnaE, Csp(CCY0110)DnaE, Csp(PCC8801)DnaE, CwaDnaE, Maer(NIES843)DnaE, Mcht(PCC7420)DnaE, MtuRecAΔ300, NspDnaE, OliDnaE, Sel(PC7942)DnaE, SspDnaE, Ssp(PCC7002)DnaE, TerDnaE-3, TelDnaE, TvuDnaE, NeqPol, TerThyXΔ132, or combinations thereof. An amino acid sequence of the N-terminal fragment of an intein can comprise SEQ ID NO:3. An amino acid sequence of the C-terminal fragment of an intein can comprise SEQ ID NO:4. The reverse transcriptase can be an M-MLV reverse transcriptase, a Marathon reverse transcriptase, a Rous sarcoma virus reverse transcriptase, an HIV-1 reverse transcriptase, an AMV reverse transcriptase, a telomerase reverse transcriptase, or any variant thereof. The first polynucleotide molecule can comprise one or more nuclear localization signals and the second polynucleotide molecule can comprise one or more nuclear localization signals. The first polynucleotide molecule can further comprise a linker. The linker can encode a polypeptide molecule comprising SEQ ID NO:22, 23, 24, 25, or 26. The first polynucleotide molecule can comprise one or more polynucleotides encoding a protein tag and the second polynucleotide molecule can comprise one or more polynucleotides encoding a protein tag.

Another embodiment provides a system for prime editing comprising: a first vector comprising a first polynucleotide molecule encoding a reverse transcriptase and an N-terminal fragment of a dimerization protein; and a second vector comprising a second polynucleotide molecule encoding a C-terminal fragment of a dimerization protein and a Cas protein.

The Cas protein can be a Cas nickase, Cas9 nickase, a dead Cas protein, a dead Cas9, or an active Cas protein.

An embodiment provides a prime editor comprising: a first polynucleotide molecule encoding a reverse transcriptase, an N-terminal fragment of Cas protein, and an N-terminal fragment of a dimerization protein; and a second polynucleotide molecule encoding a C-terminal fragment of a dimerization protein and a C-terminal fragment of Cas protein.

The N-terminal fragment of the Cas protein and the C-terminal fragment of the Cas protein, when combined, can form a full-length Cas protein. The N-terminal fragment of a dimerization protein can be the N-terminal fragment of an intein. The C-terminal fragment of a dimerization protein can be the C-terminal fragment of an intein. The Cas protein can be a Cas nickase, a Cas9 nickase, a dead Cas protein, a dead Cas9, or an active Cas protein. The Cas9 nickase can be split into a N-terminal fragment and a C-terminal fragment at a split point. The split point can be localized at any amino acid between position 564 and 584 (e.g. 564, 565, 566, 567, 568, 569, 570, 571, 572, 573, 574, 575, 576, 577, 578, 579, 580, 581, 582, 583, 584), and the N-terminal fragment of Cas9 nickase can comprise nucleotides from position 1 of a Cas9 nickase to the split point and the C-terminal fragment of Cas9 nickase can comprise nucleotides from the split point to position 1371 of a Cas9 nickase. The split point can be localized at any amino acid between position 249 and 269 (e.g., 249, 250, 251, 252, 253, 254, 255, 256, 257, 258, 259, 260, 261, 262, 263, 264, 265, 266, 267, 268, 269), and the N-terminal fragment of Cas9 nickase can comprise nucleotides from position 1 of a Cas9 nickase to the split point and the C-terminal fragment of Cas9 nickase can comprise nucleotides from the split point to position 1371 of a Cas9 nickase. The split point can be localized at any amino acid between position 265 and 285 (e.g., 265, 266, 267, 268, 269, 270, 271, 272, 273, 274, 275, 276, 277, 278, 279, 280, 281, 282, 283, 284, 285), and the N-terminal fragment of Cas9 nickase can comprise nucleotides from position 1 of a Cas9 nickase to the split point and the C-terminal fragment of Cas9 nickase can comprise nucleotides from the split point to position 1371 of a Cas9 nickase. The first polynucleotide molecule and the second polynucleotide molecule can each comprise a promoter. The Cas9 nickase can be a Cas9 protein having an amino acid substitution at position 10 or at position 840 or at position 863. The Cas9 nickase can be D10A Cas9, D10N Cas9, H840N Cas9, H840Y Cas9, H840A Cas9, or N863A Cas9. The C-terminal fragment of an intein and the N-terminal fragment of an intein can be derived from PhoRadA, RmaDnaB^{Δ286} SspDnaB^{Δ275}, SspDnaB^{M86Δ275}, SspDnaX, TvoVMA, NpuDnaE, NpuDnaB^{Δ283}, SspGyrB, AceL-TerL, PchPRP8, PfuRIR1-1, Psp-GDBPol-1, MtuRecA^{Δ228}, PfuRIR1-2, SceVMA^{Δ206}, RmaDnaB^{Δ271}, MtuRecA^{Δ285}, SspDnaB^{Δ274}, gp41-8, SceVMA^{Δ227}, IMPDH-1, NrdJ-1, MtuRecA^{Δ297}, gp41-1, AovDnaE, AspDnaE, AvaDnaE, Cra(C5505)DnaE, Csp(CCY0110)DnaE, Csp(PCC8801)DnaE, CwaDnaE, Maer(NIES843)DnaE, Mcht(PCC7420)DnaE, MtuRecA^{Δ300}, NspDnaE, OliDnaE, Sel(PC7942)DnaE, SspDnaE, Ssp(PCC7002)DnaE, TerDnaE-3, TelDnaE, TvuDnaE, NeqPol, TerThyX^{Δ132}, or combinations thereof. The reverse transcriptase can be an M-MLV reverse transcriptase, Marathon reverse transcriptase, a Rous sarcoma virus reverse transcriptase, an HIV-1 reverse transcriptase, an AMV reverse transcriptase, a telomerase reverse transcriptase, or any variant thereof. The first polynucleotide molecule can comprise one or more nuclear localization signals and the second polynucleotide molecule can comprise one or more nuclear localization signals. The sequence of the first polynucleotide molecule can encode a polypeptide comprising SEQ ID NO:5. An amino acid sequence of the N-terminal fragment of an intein can comprise SEQ ID NO:3. The sequence of the second polynucleotide molecule can encode a polypeptide comprising SEQ ID NO:6. An amino acid sequence of the C-terminal fragment of an intein can comprise SEQ ID NO:7. The first polynucleotide molecule can further comprise a linker. The linker can encode a polypeptide molecule comprising SEQ ID NO:22, 23, 24, 25, or 26. The first polynucleotide molecule can comprise one or more polynucleotides encoding a protein tag and the second polynucleotide molecule can comprise one or more polynucleotides encoding a protein tag.

Another embodiment provides a system for prime editing comprising: a first vector comprising a first polynucleotide molecule encoding a reverse transcriptase, an N-terminal fragment of Cas protein, and an N-terminal fragment of a dimerization protein; and a second vector comprising a second polynucleotide molecule encoding a C-terminal fragment of a dimerization protein and a C-terminal fragment of Cas protein.

The N-terminal fragment of the Cas protein and the C-terminal fragment of the Cas protein, when combined, can form a full-length Cas protein. The Cas protein can be a Cas nickase, a Cas9 nickase, a dead Cas protein, a dead Cas9, or an active Cas protein. The N-terminal fragment of a dimerization protein can be the N-terminal fragment of an intein. The C-terminal fragment of a dimerization protein can be the C-terminal fragment of an intein.

An embodiment provides a prime editor comprising: a first polynucleotide molecule encoding a N-terminal fragment of a Cas protein, and a N-terminal fragment of a dimerization protein; and a second polynucleotide molecule encoding a C-terminal fragment of a dimerization protein, a C-terminal fragment of Cas protein, and a reverse transcriptase.

The N-terminal fragment of the Cas protein and the C-terminal fragment of the Cas protein can, when combined, form a full-length Cas protein. The N-terminal fragment of a dimerization protein can be the N-terminal fragment of an intein. The C-terminal fragment of a dimerization protein can be the C-terminal fragment of an intein. The Cas protein can be a Cas nickase, a Cas9 nickase, a dead Cas protein, a dead Cas9, or an active Cas protein. The Cas9 nickase can be split into a N-terminal fragment and a C-terminal fragment at a split point. The split point can be localized at any amino acid between position 703 and 723, and the N-terminal fragment of Cas9 nickase can comprise nucleotides from position 1-713 of the Cas9 nickase to the split point and the C-terminal fragment of Cas9 nickase can comprise nucleotides from the split point to position 714-1371 of the Cas9 nickase. The split point can be localized at any amino acid between position 935 and 965 (e.g., 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965), and the N-terminal fragment of Cas9 nickase can comprise nucleotides from position 1-945 of the Cas9 nickase to the split point and the C-terminal fragment of Cas9 nickase can comprise nucleotides from the split point to position 946-1371 of the Cas9 nickase. The split point can be localized at any amino acid between position 1044 and 1064 (e.g., 1044, 1045, 1046, 1047, 1048, 1049, 1050, 1051, 1052, 1053, 1054, 1055, 1056, 1057, 1058, 1059, 1060, 1061, 1062, 1063, 1064) and, the N-terminal fragment of Cas9 nickase can comprise nucleotides from position 1-1054 of the Cas9 nickase to the split point and the C-terminal fragment of Cas9 nickase can comprise nucleotides from the split point to position1055-1371 of the Cas9 nickase. The split point can be localized at any amino acid between position 1105 and 1125 (e.g., 1105, 1106, 1107, 1108, 1109, 1110, 1111, 1112, 1113, 1114, 1115, 1116, 1117, 1118, 1119, 1120, 1121, 1122, 1123, 1124, 1125), and the N-terminal fragment of Cas9 nickase can comprise nucleotides from position 1-1115 of the Cas9 nickase to the split point and the C-terminal fragment of Cas9 nickase can comprise nucleotides from the split point to position1116-1371 of the Cas9 nickase. The first polynucleotide molecule and the second polynucleotide molecule can each comprise a promoter. The Cas9 nickase can be a Cas9 protein having an amino acid substitution at position 10, at position 840, or at position 863. The Cas9 nickase can be D10A Cas9, D10N Cas9, H840N Cas9, H840Y Cas9, H840A Cas9, or N863A Cas9. The C-terminal fragment of a dimerization protein and the N-terminal fragment of a dimerization protein can be derived from PhoRadA, RmaDnaBΔ286, SspDnaBΔ275, SspDnaBM86Δ275, SspDnaX, TvoVMA, NpuDnaE, NpuDnaBΔ283, SspGyrB, AceL-TerL, PchPRP8, PfuRIR1-1, Psp-GDBPol-1, MtuRecAΔ228, PfuRIR1-2, SceVMAΔ206, RmaDnaBΔ271, MtuRecAΔ285, SspDnaBΔ274, gp41-8, SceVMAΔ227, IMPDH-1, NrdJ-1, MtuRecAΔ297, gp41-1, AovDnaE, AspDnaE, AvaDnaE, Cra(C5505)DnaE, Csp(CCY0110)DnaE, Csp(PCC8801)DnaE, CwaDnaE, Maer(NIES843)DnaE, Mcht(PCC7420)DnaE, MtuRecAΔ300, NspDnaE, OliDnaE, Sel(PC7942)DnaE, SspDnaE, Ssp(PCC7002)DnaE, TerDnaE-3, TelDnaE, TvuDnaE, NeqPol, TerThyXΔ132, or combinations thereof. The reverse transcriptase is an M-MLV reverse transcriptase, Marathon reverse transcriptase, a Rous sarcoma virus reverse transcriptase, an HIV-1 reverse transcriptase, an AMV reverse transcriptase, a telomerase reverse transcriptase, or any variant thereof. The first polynucleotide molecule can comprise one or more nuclear localization signals and the second polynucleotide molecule can comprise one or more nuclear localization signals. The sequence of the first polynucleotide molecule can encode a polypeptide comprising SEQ ID NO:9, 11, 13, or 15. An amino acid sequence of the N-terminal fragment of an intein can comprise SEQ ID NO:3. The sequence of the second polynucleotide molecule can encode a polypeptide comprising SEQ ID NO:10, 12, 14, or 16. An amino acid sequence of the C-terminal fragment of an intein can comprise SEQ ID NO:7. The first polynucleotide molecule can further comprise a linker. The linker can encode a polypeptide molecule comprising SEQ ID NO:22, 23, 24, 25, or 26. The first polynucleotide molecule can comprise one or more polynucleotides encoding a protein tag and the second polynucleotide molecule can comprise one or more polynucleotides encoding a protein tag.

Another embodiment provides a system for prime editing comprising: a first vector comprising a first polynucleotide molecule encoding a N-terminal fragment of a Cas protein, and a N-terminal fragment of a dimerization protein; and a second vector comprising a second polynucleotide molecule encoding a C-terminal fragment of a dimerization protein, a C-terminal fragment of Cas protein, and a reverse transcriptase.

The N-terminal fragment of the Cas protein and the C-terminal fragment of the Cas protein can, when combined, form a full-length Cas protein. The N-terminal fragment of a dimerization protein can be the N-terminal fragment of an intein. The C-terminal fragment of a dimerization protein can be the C-terminal fragment of an intein. The Cas protein can be a Cas nickase, a Cas9 nickase, a dead Cas protein, a dead Cas9, or an active Cas protein.

An embodiment provides a method of editing genomic DNA in a cell comprising contacting the cell with a system for prime editing.

The system can further comprise one or more pegRNA molecules, one or more sgRNA molecules, or a combination of one or more pegRNA molecules and one or more sgRNA molecule. The one or more pegRNA molecule can comprise one or more loops, one or more base modifications, or a combination of one or more loops and one or more base modifications to enhance prime editing activity. Prime editing genomic DNA can avoid generating a double-stranded break. Editing genomic DNA can induce an insertion, deletion, transversion point mutation, or transition point mutation. A first vector and a second vector can be AAV vectors. The one or more pegRNA molecules can comprise SEQ ID NOs:17, 18, 19, 20, or 21.

Provided herein are split prime editors, systems for prime editing, methods of editing genomic DNA in a cell using systems comprising split prime editors.

### BRIEF DESCRIPTION OF THE DRAWINGS

The features, objects and advantages other than those set forth above will become more readily apparent when consideration is given to the detailed description below. Such detailed description makes reference to the following drawings, wherein:

FIG. 1 illustrates a test system implemented to identify efficient prime editors. GCCGAGGTGTAGTTCGAGGGC is SEQ ID NO: 29; CGGCTCCACATCAAGC TCCCG SEQ ID NO:30; GCCGAGGTGAAGTTCGAGGGC is SEQ ID NO:31; CGGCTCCACTTCAAGCTCCCG SEQ ID NO:32; and AEVKFEG is SEQ ID NO:35.

FIG. 2 is a graph bar illustrating the percentage of GFP positive cells obtained with different pegRNA and proxyRNA combinations.

FIG. 3A illustrates constructs of a prime editor with split point at amino acid 945 of Cas9.

FIG. 3B illustrates constructs of a prime editor with split point between Cas9 and the reverse transcriptase (RT).

FIG. 3C illustrates prime editing constructs with the reverse transcriptase at the C-terminus or at the N-terminus (N-MMLV).

FIG. 3D illustrates constructs of a prime editor using a marathon reverse transcriptase.

FIG. 4 is a graph bar illustrating the percentage of activity of the constructs relative to the full length prime editors.

FIG. 5 illustrates the percentage of activity of a prime editor using marathon reverse transcriptase relative to a prime editor using MMLV reverse transcriptase.

FIG. 6A illustrates the modification rate in genomic DNA by Sanger sequencing using a wild type (WT) prime editor. AAGGGCCTGAGTCCGAGCAG AAGAAGAAGGGCTCCCATCACATCAAC is SEQ ID NO:33; AAGGGCCTGAGT CCGAGCAGAAGAAGAAGNGCTCCCATCACATCAAC is SEQ ID NO:34 (N is any nucleotide); GAGCAGAAGAAGAAGGGCTCCC is SEQ ID NO:36.

FIG. 6B illustrates the modification rate in genomic DNA by Sanger sequencing using a prime editor with the split point between Cas9 and the reverse transcriptase (RT). AAGGGCCTGAGTCCGAGCAGAAGAAGAAGGGCTCCCATC ACATCAAC is SEQ ID NO:33; AAGGGCCTGAGTCCGAGCAGAAGAAGAAGNGC TCCCATCACATCAAC SEQ ID NO:34 (N is any nucleotide); CAGAAGAAGAAGGGC TCCC is SEQ ID NO:37.

FIG. 6C illustrates the modification rate in genomic DNA by Sanger sequencing using N-term PE prime editor. AAGGGCCTGAGTCCGAGCAG AAGAAGAAGGGCTCCCATCACATCAAC is SEQ ID NO:33; AAGGGCCTGAGT CCGAGCAGAAGAAGAAGNGCTCCCATCACATCAAC is SEQ ID NO:34 (N is any nucleotide); GAGCAGAAGAAGAAGGGCTCCC SEQ ID NO:36.

FIG. 6D illustrates the modification rate in genomic DNA by Sanger sequencing using split prime editor with the reverse transcriptase (RT) at the N-terminus.

AAGGGCCTGAGTCCGAGCAGAAGAAGAAGGGCTCCCATCACATCAACCG
GTGGCG is SEQ ID NO: 38;
CAGAAGAAGAAGGGCTCCC is SEQ ID NO: 37.

FIG. 7A illustrates the various pegRNA constructs that use two sgRNA scaffolds.

CGATTTCTTGGCTTTATATATCTTGTGGAAAGGACGAAACACCGGCAA
CTACAAGACCCGCGCCGGTTTTAGAGCTAGAAATAGCAAGTTAAAATA
AGGCTAGTCCGTTATCAACTTGAAAAAGTGGCACCGAGTCGGTGCCCT
CGAACTtCACCTCGGCGCGGGTCTTTTTTTT is SEQ ID
NO: 17;
GCTAAAGAACCGAAATATATAGAACACCTTTCCTGCTTTGTGGCCGTT
GATGTTCTGGGCGCGGCCAAAATCTCGATCTTTATCGTTCAATTTTAT
TCCGATCAGGCAATAGTTGAACTTTTTCACCGTGGCTCAGCCACGGGA
GCTTGAaGTGGAGCCGCGCCCAGAAAAAAAA is SEQ ID
NO: 39;
CGATTTCTTGGCTTTATATATCTTGTGGAAAGGACGAAACACCGGCAA
CTACAAGACCCGCGCCGGTTTTAGAGCTAGAAATAGCAAGTTAAAATA
AGGCTAGTCCGTTATCAACTTGAAAAAGTGGCACCGAGTCGGTGCCCT
CGAACTtCACCTCGGCGCGGGTCTGTTTTAGAGCTAGAAATAGCAAGT
TAAAATAAGGCTAGTCCGTTATCAACTTGAAAAAGTGGCACCGAGTCG
GTGCTTTTTTT is SEQ ID NO: 18;
GCTAAAGAACCGAAATATATAGAACACCTTTCCTGCTTTGTGGCCGTT
GATGTTCTGGGCGCGGCCAAAATCTCGATCTTTATCGTTCAATTTTAT
TCCGATCAGGCAATAGTTGAACTTTTTCACCGTGGCTCAGCCACGGGA
GCTTGAAGTGGAGCCGCGCCCAGACAAAATCTCGATCTTTATCGTTCA
ATTTTATTCCGATCAGGCAATAGTTGAACTTTTTCACCGTGGCTCAGC
CACGAAAAAAA is SEQ ID NO: 40;
CGATTTCTTGGCTTTATATATCTTGTGGAAAGGACGAAACACCGGCAA
CTACAAGACCCGCGCCGGTTTTAGAGCTAGAAATAGCAAGTTAAAATA
AGGCTAGTCCGTTATCAACTTGAAAAAGTGGCACCGAGTCGGTGCCCT
CGAACTtCACCTCGGCGCGGGTCTTTTTTAGAGCTAGAAATAGCAAGT
TAAAATAAGGCTAGTCCGTTATCAACTTGAAAAAGTGGCACCGAGTCG
GTGCTTTTTTT is SEQ ID NO: 19;
GCTAAAGAACCGAAATATATAGAACACCTTTCCTGCTTTGTGGCCGTT
GATGTTCTGGGCGCGGCCAAAATCTCGATCTTTATCGTTCAATTTTAT
TCCGATCAGGCAATAGTTGAACTTTTTCACCGTGGCTCAGCCACGGGA
GCTTGAaGTGGAGCCGCGCCCAGAAAAAATCTCGATCTTTATCGTTCA
ATTTTATTCCGATCAGGCAATAGTTGAACTTTTTCACCGTGGCTCAGC
CACGAAAAAAA is SEQ ID NO: 41;
CGATTTCTTGGCTTTATATATCTTGTGGAAAGGACGAAACACCGGCAA
CTACAAGACCCGCGCCGGTTTTAGAGCTAGAAATAGCAAGTTAAAATA
AGGCTAGTCCGTTATCAACTTGAAAAAGTGGCACCGAGTCGGTCCTCG
AACTtCACCTCGGCGCGGGTCTGTTTTAGAGCTAGAAATAGCAAGTTA
AAATAAGGCTAGTCCGTTATCAACTTGAAAAAGTGGCACCGAGTCGGT
GCTTTTTTT is SEQ ID NO: 20;
GCTAAAGAACCGAAATATATAGAACACCTTTCCTGCTTTGTGGCCGTT
GATGTTCTGGGCGCGGCCAAAATCTCGATCTTTATCGTTCAATTTTAT
TCCGATCAGGCAATAGTTGAACTTTTTCACCGTGGCTCAGCCAGGAGC
TTGAaGTGGAGCCGCGCCCAGACAAAATCTCGATCTTTATCGTTCAAT
TTTATTCCGATCAGGCAATAGTTGAACTTTTTCACCGTGGCTCAGCCA
CGAAAAAAA is SEQ ID NO: 42;
CGATTTCTTGGCTTTATATATCTTGTGGAAAGGACGAAACACCGGCAA
CTACAAGACCCGCGCCGGTTTTAGAGCTAGAAATAGCAAGTTAAAATA
AGGCTAGTCCGTTATCAACTTGAAAAAGTGGCACCGAGTCGGTCCTCG
AACTtCACCTCGGCGCGGGTCTTTTTTAGAGCTAGAAATAGCAAGTTA
AAATAAGGCTAGTCCGTTATCAACTTGAAAAAGTGGCACCGAGTCGGT
GCTTTTTTT is SEQ ID NO: 21;
and
GCTAAAGAACCGAAATATATAGAACACCTTTCCTGCTTTGTGGCCGTT
GATGTTCTGGGCGCGGCCAAAATCTCGATCTTTATCGTTCAATTTTAT
TCCGATCAGGCAATAGTTGAACTTTTTCACCGTGGCTCAGCCAGGAGC
TTGAaGTGGAGCCGCGCCCAGAAAAAATCTCGATCTTTATCGTTCAAT
TTTATTCCGATCAGGCAATAGTTGAACTTTTTCACCGTGGCTCAGCCA
CGAAAAAAA is SEQ ID NO: 43.

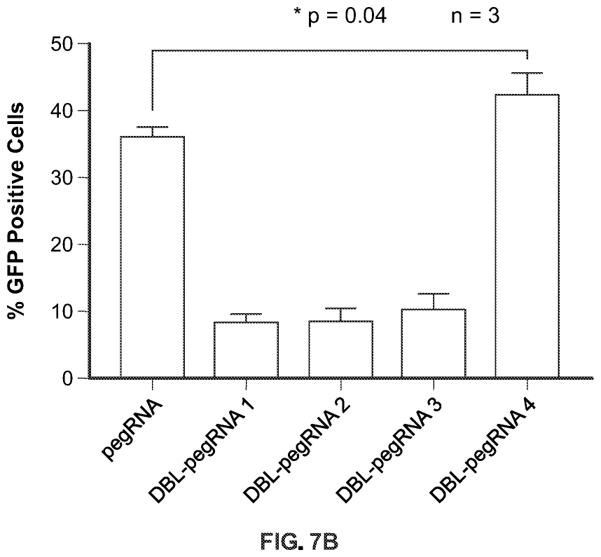

FIG. 7B is a graph bar showing the % GFP+ cells obtained after using the modified pegRNA constructs with the GFP reporter system.

### DETAILED DESCRIPTION

Many modifications and other embodiments of prime editors and methods of use thereof described herein will come to mind to one of skill in the art having the benefit of the teachings presented in the foregoing descriptions and the associated drawings. Therefore, it is to be understood that the methods and compositions are not to be limited to the specific embodiments disclosed and that modifications and other embodiments are intended to be included within the scope of the appended claims.

Although specific terms are employed herein, they are used in a generic and descriptive sense only and not for purposes of limitation.

Unless defined otherwise, all technical and scientific terms used herein have the same meaning as commonly understood by one of skill in the art.

### Overview

Prime editing is a genome editing technology that can directly write new genetic information into a targeted DNA site. Using a fusion protein comprising a catalytically impaired Cas endonuclease and an engineered reverse transcriptase enzyme, along with a prime editing guide RNA (pegRNA), capable of identifying a target site, and a single guide RNA (sgRNA) which nicks the other strand, new genetic information can be provided at the target site, and target DNA nucleotides can be replaced. Prime editing can mediate targeted insertions, deletions, and base-to-base conversions (transversion and transition) without generating double strand breaks (DSBs) and without requiring donor DNA templates.

Prime editing involves three major components: (1) a prime editing guide RNA (pegRNA) that can identify of a target nucleotide sequence to be edited and that encodes the genetic information to incorporate at the targeted sequence. pegRNA comprises an extended single guide RNA (sgRNA) containing a primer binding site (PBS) and a reverse transcriptase (RT) template sequence; (2) a fusion protein comprising a Cas protein (e.g., a H840A nickase) fused to a reverse transcriptase (e.g., Moloney Murine Leukemia Virus (M-MLV) reverse transcriptase); and (3) an optional single guide RNA (sgRNA) that directs the Cas protein portion of the fusion protein to nick the non-edited DNA strand.

A fusion protein nicks the target DNA sequence, that can be used to initiate (prime) the reverse transcription of the RT template portion of the pegRNA. The reannealed double stranded DNA contains nucleotide mismatches at the location where the RT template differs from the genomic sequence. To correct the mismatches, the cells exploit the intrinsic mismatch repair mechanism, with two possible outcomes: (i) the information in the edited strand is copied into the complementary strand, permanently installing the edit; (ii) the original nucleotides are re-incorporated into the edited strand, excluding the edit.

By relying on DNA mismatch repair instead of non-homologous end joining (NHEJ) or homology-directed repair (HDR) to fix DNA breaks, prime editing avoids the generation of DSBs.

Despite their considerable potential for treating human diseases, the implementation of prime editors for in vivo gene correction is limited by the fact that their size exceeds the carrying capacity of a single commonly used vector (e.g. an AAV vector). Described herein are split prime editors that overcome the packaging limitations associated with delivering prime editors and that can be used for treating human diseases.

To overcome the packaging limitations associated with delivering prime editors, prime editors can be split into two different domains, each of which can be fused with an intein-based trans-splicing system. Following delivery to cells and expression, the inteins can bind to each other and excise themselves out while creating a peptide bond between the two prime editor domains that reconstitutes the full-length prime editor protein.

An embodiment provides a prime editor comprising a first polynucleotide molecule encoding a Cas protein and an N-terminal fragment of a dimerization protein; and a second polynucleotide molecule encoding a C-terminal fragment of a dimerization protein and a reverse transcriptase.

A prime editor can comprise a first polynucleotide comprising a reverse transcriptase and an N-terminal fragment of a dimerization protein, and a second polynucleotide molecule encoding a C-terminal fragment of a dimerization protein and a Cas protein. A prime editor can be split so that a first domain comprises a Cas protein and an N-terminal fragment of a dimerization protein, and a second domain comprises a reverse transcriptase and a C-terminal fragment of a dimerization protein. Upon interaction and self-excision of the dimerization proteins, a fusion protein comprising a Cas protein and a reverse transcriptase can be reconstituted, and prime editing can occur.

Polynucleotides and Polypeptides

Polynucleotides refer to nucleic acid molecules comprising deoxyribonucleic acid (DNA) or ribonucleic acid (RNA). Nucleic acid molecules include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated, oligonucleotides or combinations thereof. Polynucleotides can be present as a single-stranded or double-stranded and linear or covalently circularly closed molecule.

Polynucleotides can be obtained from nucleic acid molecules present in, for example, a mammalian cell. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides can be isolated. An isolated polynucleotide can be a naturally-occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid molecules naturally found immediately flanking the recombinant DNA molecule in a naturally-occurring genome is removed or absent. Isolated polynucleotides also include non-naturally occurring nucleic acid molecules. Polynucleotides can encode full-length polypeptides, polypeptide fragments, and variant or fusion polypeptides. “Isolated polynucleotides” can be (i) amplified in vitro, for example via polymerase chain reaction (PCR), (ii) produced recombinantly by cloning, (iii) purified, for example, by cleavage and separation by gel electrophoresis, (iv) synthesized, for example, by chemical synthesis, or (v) extracted from a sample.

A polynucleotide can comprise, for example, a gene, open reading frame, non-coding region, or regulatory element. A gene is any polynucleotide molecule that encodes a polypeptide, protein, or fragment thereof, optionally including one or more regulatory elements preceding (5′ non-coding sequences) and following (3′ non-coding sequences) the coding sequence. In one embodiment, a gene does not include regulatory elements preceding and following the coding sequence. A native or wild-type gene refers to a gene as found in nature, optionally with its own regulatory elements preceding and following the coding sequence. A chimeric or recombinant gene refers to any gene that is not a native or wild-type gene, optionally comprising regulatory elements preceding and following the coding sequence, wherein the coding sequences and/or the regulatory elements, in whole or in part, are not found together in nature. Thus, a chimeric gene or recombinant gene comprise regulatory elements and coding sequences that are derived from different sources, or regulatory elements and coding sequences that are derived from the same source, but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide. Polynucleotides can encode the polypeptides described herein (e.g., a Cas9 nickase, or a fragment thereof, a N-terminal fragment of an intein, a C-terminal fragment of an intein, or a reverse transcriptase).

Degenerate polynucleotide sequences encoding polypeptides described herein, as well as homologous nucleotide sequences are contemplated herein. A homologous nucleotide sequence can be at least about 30, 40, 50, 60, 70, 80, or about 85, 90, 91, 92, 93, 94, 95, 96, 97, 98, or 99% identical to polynucleotides described herein and the complements thereof are also polynucleotides. Degenerate nucleotide sequences are polynucleotides that encode a polypeptide described herein or fragments thereof, but differ in nucleic acid sequence from the wild-type polynucleotide sequence, due to the degeneracy of the genetic code. Complementary DNA (cDNA) molecules, species homologs, and variants of polynucleotides that encode biologically functional polypeptides also are polynucleotides.

Polynucleotides can comprise coding sequences for naturally occurring polypeptides or can encode altered sequences that do not occur in nature.

Unless otherwise indicated, the term polynucleotide or gene includes reference to the specified sequence as well as the complementary sequence thereof.

The expression products of genes or polynucleotides are often proteins, or polypeptides, but in non-protein coding genes such as rRNA genes or tRNA genes, the product is a functional RNA. The process of gene expression is used by all known life forms, i.e., eukaryotes (including multicellular organisms), prokaryotes (bacteria and archaea), and viruses, to generate the macromolecular machinery for life. Several steps in the gene expression process can be modulated, including the transcription, up-regulation, RNA splicing, translation, and post-translational modification of a protein.

A polypeptide is a polymer of two or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide, etc., has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure. A purified polypeptide does not include unpurified or semi-purified cell extracts or mixtures of polypeptides that are less than 70% pure.

The term “polypeptides” can refer to one or more of one type of polypeptide (a set of polypeptides). “Polypeptides” can also refer to mixtures of two or more different types of polypeptides (a mixture of polypeptides). The terms “polypeptides” or “polypeptide” can each also mean “one or more polypeptides.”

As used herein, the term “polypeptide of interest” or “polypeptides of interest”, “protein of interest”, “proteins of interest” includes any or a plurality of any of the Cas proteins or fragments thereof, N-terminal fragments of dimerization protein, C-terminal fragments of dimerization protein, reverse transcriptase polypeptides, linkers, protein tags, or other polypeptides (including fragment polypeptides) described herein.

A mutated protein or polypeptide comprises at least one deleted, inserted, and/or substituted amino acid, which can be accomplished via mutagenesis of polynucleotides encoding these amino acids. Mutagenesis includes well-known methods in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989).

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar Variants will be sufficiently similar to the amino acid sequence of the polypeptides described herein. Such variants generally retain the functional activity of the polypeptides described herein. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity.

Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

Polypeptides and polynucleotides that are sufficiently similar to polypeptides and polynucleotides described herein (e.g., Cas proteins, dimerization protein, reverse transcriptase polypeptides, linkers, protein tags, or polypeptide fragments thereof) can be used herein. Polypeptides and polynucleotides that are about 85, 90, 91, 92, 93, 94 95, 96, 97, 98, 99 99.5% or more homologous or identical to polypeptides and polynucleotides described herein (e.g., Cas proteins, dimerization proteins, reverse transcriptase polypeptides, linkers, protein tags, or polypeptide fragments thereof) can also be used herein.

A prime editor can comprise a first polynucleotide molecule encoding one or more nuclear localization signals (NLS), a Cas protein, and an N-terminal fragment of a dimerization protein. The Cas nickase can be a Cas9 nickase. For example, a first polynucleotide can comprise two NLSs, a Cas9 nickase, and an N-terminal fragment of an intein (e.g., SEQ ID NO:3). In an embodiment, a first polynucleotide can comprise SEQ ID NO:1.

A prime editor can comprise a second polynucleotide molecule encoding one or more nuclear localization signals (NLS), a reverse transcriptase, and a C-terminal fragment of an intein. For example a second polynucleotide can comprise two NLSs, a reverse transcriptase, and C-terminal fragment of an intein (e.g., SEQ ID NO:4). In an embodiment, a second polynucleotide can comprise SEQ ID NO:2.

In an embodiment, a prime editor can comprise a first polynucleotide comprising SEQ ID NO:1 and a second polynucleotide comprising SEQ ID NO:2.

Cas Proteins

A “catalytically active RNA-guided DNA endonuclease protein,” or “DNA endonuclease” refers to an endonuclease protein directed to a specific DNA target by a gRNA, where it causes a double-strand break. There are many versions of RNA-guided DNA endonucleases isolated from different organisms. Each RNA-guided DNA endonuclease binds to its target sequence in the presence of a protospacer adjacent motif (PAM), on the non-targeted DNA strand. Therefore, the locations in a genome that can be targeted by different RNA-guided DNA endonucleases can be dictated by locations of PAM sequences. An RNA-guided DNA endonuclease can generate either a blunt or a sticky ended cut at its target site. Recognition of the PAM sequence by an RNA-guided DNA endonuclease protein is thought to destabilize the adjacent DNA sequence, allowing interrogation of the sequence by the sgRNA, and allowing the sgRNA-DNA pairing when a matching sequence is present. While the PAM sequence itself is necessary for cleavage, it is not included in the single guide RNA sequence.

Additional enzymes, such as the PAM-less or near-PAM-less SpRY Cas9, any variants of the endonucleases, high-fidelity endonucleases, or endonucleases with modified PAM requirements can be used as a split prime editor. Binding of an RNA-guided DNA endonuclease to its target sequence can thus happen in the absence of a protospacer adjacent motif (PAM).

Methods and compositions described herein can comprise a Cas proteins, such as Cas9 nickase. Cas9 nickases comprise only one catalytically active domain (either the HNH domain or the RuvC domain). Cas9 nickases retain DNA binding based on gRNA specificity, but are capable of cutting only one strand of DNA resulting in a single-strand break (e.g. a “nick”).

Cas (CRISPR associated protein) proteins are RNA-guided DNA endonuclease enzymes associated with the Clustered Regularly Interspaced Short Palindromic Repeats (CRISPR), widely used in genetic engineering applications, as they can be used to induce site-directed double-strand breaks in DNA based on the complementarity to the guide RNA. RNA-guided Cas enzymes can be dual (e.g., Cas9, Cas12b) and have a 2-part guide RNA in the native system, as opposed to single-RNA guided ones (e.g., Cas12a). A Cas nuclease can be mutated in a variety of ways to improve specificity and control. Nuclease domains can be mutated independently of each other to generate Cas nickases, which have one active and inactive nuclease domain; and which results in a complex that performs single strand cleavage. A Cas enzyme can be a Cas endonuclease Dead (also known as dead Cas or dCas), a mutant form of the protein whose endonuclease activity is removed through point mutations in its endonuclease domains. Any Cas enzyme can be modified to generate a dead Cas protein.

Non-limiting examples of RNA-guided DNA endonuclease proteins include Cas1, Cas1 B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cas-Phi, homologs thereof, variants thereof, or modified versions thereof, such as modification to generate nickases.

The range of sequences recognized by Cas nucleases is constrained by the need for a specific protospacer adjacent motif (PAM). For example, Cas from different bacterial species can recognize different PAM sequences. For example, the SpCas9 nuclease cuts upstream of the PAM sequence 5′-NGG-3′ (where “N” can be any nucleotide base), while the PAM sequence 5′-NNGRR(N)-3′ (where “N” can be any nucleotide base and “R” can be either A or G) is required for SaCas9 (from Staphylococcus aureus) to target a DNA region for editing. While the PAM sequence itself is necessary for cleavage, it is not included in the single guide RNA sequence.

As a result, the engineering of Cas derivatives with purposefully altered PAM specificities address this limitation. Such Cas enzymes (i.e., PAM modified Cas), can also be used in the prime editor described herein.

High fidelity Cas enzymes, with improved specificity, developed to reduce the frequency of off-target events associated with wild type Cas can also be used in the prime editors described herein.

Methods and compositions described herein can comprise a Cas protein. The Cas protein can be a Cas nickase, a Cas9 nickase, a dead Cas protein, a dead Cas9, or an active Cas protein. Cas9 nickases comprise only one catalytically active domain (either the HNH domain or the RuvC domain). Cas9 nickases retain DNA binding based on gRNA specificity, but are capable of cutting only one strand of DNA resulting in a single-strand break (e.g. a “nick”).

In an embodiment, the catalytically active RNA-guided DNA endonuclease protein can be a CRISPR associated protein 9 (Cas9) nickase. The Cas9 nickase can a Cas9 protein having an amino acid substitution at position 10 or at position 840 or at position 863. Any amino acid substitution that removes the aspartic acid at position 10 (D10), as well as any amino acid substitution that removes the histidine at position 840 (H840) can be used to alter the catalytic activity of the enzyme. For example, the introduction of a H840A substitution in a Cas9 nuclease, through which the 840 amino acid histidine is replaced by an alanine, inactivates one of the nuclease domains. With only one functioning domain, the catalytically impaired Cas9 (H840A Cas9) can only introduce a single strand nick.

Various alterations of Cas9 can lead to the generation of a Cas9 nickase; non-limiting examples of Cas9 nickases include D10A Cas9, D10N Cas9, H840N Cas9, H840Y Cas9, H840A Cas9, or N863A Cas9. Equivalent modifications can be applied to Cas9 variants, as well as to any alternative Cas protein.

Dimerization Proteins

Dimerization proteins are intron-like proteins that can splice proteins. Dimerization proteins can spontaneously excise itself from two host proteins or protein domains and splice its flanking N- and C-terminal domains to become a mature protein. Protein splicing is a post-translational process that includes peptide bond cleavage and backbone conjugation activities, which requires no cofactor or ATP hydrolysis.

Non limiting examples of dimerization proteins include inteins, inducible dimers, or other non-inteins. In one embodiment, the dimerization protein is an intein. Similar split system using FKBP/FRB, SNAPtag/HaloTag, light-inducible dimers, any dimerizing protein pairs, or any other protein that can be used to enforce transient or permanent dimerization can be in place of inteins.

Inteins can be divided into three major regions, an amino (or N) terminal splicing domain (INn), a carboxy (or C) terminal splicing domain (INc), and an optional endonuclease region. The N- and C-terminal splicing domains comprise conserved amino acid motifs shared by all known inteins, and including a cysteine (or serine or threonine) residue following the scissile peptide bonds at the N-terminal and C-terminal splice junctions, as well as a highly conserved asparagine at the C-terminus of the intein. These amino acid residues appear to directly participate in the cleavage of the two flanking peptide bonds and linkage of the external protein sequences.

In an embodiment, an N-terminal fragment of an intein comprises an N-terminal splicing domain (INn). In an embodiment, a C-terminal fragment of an intein comprises a C-terminal splicing domain (INc).

Inteins can excise themselves out of the host protein while reconnecting the remaining N and C exteins (i.e., the protein previously bound to the inteins) via a new peptide bond, therefore, inteins can be used to generate fusion protein. For example, if a cell expresses a first polypeptide encoding a Cas9 nickase, and an N-terminal fragment of an intein, and a second polypeptide encoding a C-terminal fragment of an intein and a reverse transcriptase; upon translation of both proteins, the N-terminal fragment of an intein and the C-terminal fragment of an intein can perform an autocatalytic reaction to generate new bonds between the two intein fragments, excise themselves, and generate a new peptidic bond between the Cas9 nickase and the reverse transcriptase.

Many inteins are known in the art, and they can be derived from various organisms. For example, a C-terminal fragment of an intein and a N-terminal fragment of an intein can be derived from PhoRadA, RmaDnaB^{Δ286}, SspDnaB^{Δ275} SspDnaB^{M86Δ275}, SspDnaX, TvoVMA, NpuDnaE, NpuDnaB^{Δ283}, SspGyrB, AceL-TerL, PchPRP8, PfuRIR1-1, Psp-GDBPol-1, MtuRecA^{Δ228}, PfuRIR1-2, SceVMA^{Δ206}, RmaDnaB^{Δ271}, MtuRecA^{Δ285}, SspDnaB^{Δ274}, gp41-8, SceVMA^{Δ227}, IMPDH-1, NrdJ-1, MtuRecA^{Δ297}, gp41-1, AovDnaE, AspDnaE, AvaDnaE, Cra(C5505)DnaE, Csp(CCY0110)DnaE, Csp(PCC8801)DnaE, CwaDnaE, Maer(NIES843)DnaE, Mcht(PCC7420)DnaE, MtuRecA^{A300}, NspDnaE, OliDnaE, Sel(PC7942)DnaE, SspDnaE, Ssp(PCC7002)DnaE, TerDnaE-3, TelDnaE, TvuDnaE, NeqPol, or TerThyX^{Δ132}.

Reverse Transcriptase

A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses.

Retroviral RT has three sequential biochemical activities: RNA-dependent DNA polymerase activity, ribonuclease H (RNAse H), and DNA-dependent DNA polymerase activity. Collectively, these activities enable the enzyme to convert single-stranded RNA into double-stranded cDNA. In retroviruses and retrotransposons, this cDNA can then integrate into the host genome, from which new RNA copies can be made via host-cell transcription. The same sequence of reactions is widely used to convert RNA to DNA for use in molecular cloning, RNA sequencing, polymerase chain reaction (PCR), or genome analysis. Prime editor and genome editing in general rely on the use of RTases for their ability to generate DNA using an RNA template. Any RTase that synthesizes DNA from a RNA template can be used, including engineered RT, any protein that can generate DNA based on a RNA template, variants thereof and mutants thereof. Non-limiting examples or RTases include: Rous sarcoma virus reverse transcriptase; HIV-1 reverse transcriptase from human immunodeficiency virus type 1; M-MLV reverse transcriptase from the Moloney murine leukemia virus; AMV reverse transcriptase from the avian myeloblastosis; Marathon reverse transcriptase; telomerase reverse transcriptase, and any variant thereof.

A reverse transcriptase can be an engineered RTase, comprising mutations in the polynucleotide sequence of the enzyme, that are responsible for enhancing the binding of the enzyme to the template, the enzyme processivity, and the enzyme thermostability.

In an embodiment, a reverse transcriptase of a prime editor can be an M-MLV reverse transcriptase (e.g., accession number M32803) or a Marathon reverse transcriptase (e.g., SEQ ID NO:8). M-MLV reverse transcriptase is known for its ability to synthesize DNA from a single-stranded RNA template.

A reverse transcriptase can be at the N-terminus of a polynucleotide of a prime editor, or a reverse transcriptase can be at the C-terminus of a polynucleotide of a prime editor. A reverse transcriptase sequence can also be inserted in the sequence of the Cas protein, at the N-terminus or at the C-terminus of a polynucleotide.

Promoters

A promoter is a polynucleotide that is capable of controlling the expression of a coding sequence or gene. Promoters are generally located 5′ of the sequence that they regulate. Promoters can be derived in their entirety from a native gene, or be composed of different elements derived from promoters found in nature, and/or comprise synthetic nucleotide segments. Those skilled in the art will readily ascertain that different promoters can regulate expression of a coding sequence or gene in response to a particular stimulus, e.g., in a cell- or tissue-specific manner, in response to different environmental or physiological conditions, or in response to specific compounds. Promoters are typically classified into two classes: inducible and constitutive. A constitutive promoter refers to a promoter that allows for continual transcription of the coding sequence or gene under its control.

An inducible promoter refers to a promoter that initiates increased levels of transcription of the coding sequence or gene under its control in response to a stimulus or an exogenous environmental condition. If inducible, there are inducer polynucleotides present therein that mediate regulation of expression so that the associated polynucleotide is transcribed only when an inducer molecule is present. A directly inducible promoter refers to a regulatory region, wherein the regulatory region is operably linked to a gene encoding a protein or polypeptide, where, in the presence of an inducer of the regulatory region, the protein or polypeptide is expressed. An indirectly inducible promoter refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by inducible promoter.

A promoter can be any polynucleotide that shows transcriptional activity in the chosen host organism. A promoter can be naturally-occurring, can be composed of portions of various naturally-occurring promoters, or may be partially or totally synthetic. Guidance for the design of promoters is derived from studies of promoter structure, such as that of Harley and Reynolds, Nucleic Acids Res., 15, 2343-61 (1987). In addition, the location of the promoter relative to the transcription start can be optimized. Many suitable promoters for use in mammalian cells are well known in the art, as are polynucleotides that enhance expression of an associated expressible polynucleotide. Non-limiting examples of promoters that can be used to in the present expression cassette can include cytomegalovirus (CMV) promoter and the Rous sarcoma virus promoter, that allows for unregulated expression in mammalian cells.

In an embodiment, the first polynucleotide molecule and the second polynucleotide molecule can each comprise a promoter.

Nuclear Localization Signals

A nuclear localization signal or sequence (NLS) is an amino acid sequence that ‘tags’ a protein for import into the nucleus by nuclear transport. Typically, this signal comprises one or more short sequences of positively charged lysines or arginines exposed on the protein surface. Different nuclear localized proteins can share the same NLS. There are two types of NLSs, the classical and the non-classical NLS.

Classical NLSs can be classified as either monopartite or bipartite, depending on the presence of a short spacer sequence separating the two basic amino acid clusters (present in bipartite NLSs).

An example of a monopartite NLS includes PKKKRKV (SEQ ID NO:27) from the SV40 Large T-antigen; while the NLS of nucleoplasmin, KR[PAATKKAGQA]KKKK (SEQ ID NO:28) is an example of bipartite signal. Both signals can be recognized by importin α. Importin α contains a bipartite NLS itself, which is specifically recognized by importin β, considered as the actual import mediator.

Many other non-classical NLS are also known, such as the acidic M9 domain of hnRNP A1, the sequence KIPIK in yeast transcription repressor Mata2, and the complex signals of U snRNPs. Most of these NLSs appear to be recognized directly by specific receptors of the importin β family without the intervention of an importin α-like protein.

Any NLS can be used in the prime editor described herein, including inducible NLSs such as light-inducible NLSs for example.

Polynucleotide molecules described herein can include one or more NLS. For example, a polynucleotide can comprise 1, 2, 3, 4, or more NLSs of any type. For example, a polynucleotide can comprise 1, 2, 3, 4, or more NLSs, wherein the 1, 2, 3, 4, or more NLSs are classical NLSs; a polynucleotide can comprise 1, 2, 3, 4, or more NLSs, wherein the 1, 2, 3, 4, or more NLSs are non-classical NLSs; or a polynucleotide can comprise 1, 2, 3, 4, or more NLSs, some of 1, 2, 3, 4, or more of the NLSs are classical NLSs and the remaining of the 1, 2, 3, 4, or more of the NLSs are non-classical NLSs. The NLS sequence can occur anywhere in the molecule. For example, a NLS sequence can be incorporated at the 5′ end of a polynucleotide molecule, at the 3′ end of the molecule, or both at the 5′ and at the 3′ end of the molecule.

Linkers

Linkers are polynucleotide sequences than can encode a polypeptide joining the RT and the Cas protein in a prime editor. Linkers can greatly influence the activity of a prime editor.

In an embodiment polynucleotide molecule encoding a RT can further comprise a linker, so that the linker can join the RT and the Cas protein. In some aspects, the first polynucleotide molecule can further comprise a linker. In other aspects, the second polynucleotide molecule can further comprise a linker. The linker can encode a polypeptide molecule comprising SEQ ID NO:22, 23, 24, 25, or 26.

Protein Tags

Proteins tags are small polypeptide sequences that can be used for protein detection. Proteins tags do not modify activity of the prime editors, but can be used for isolation, or detection of the prime editors.

Non limiting examples of protein tags include V5, His, or FLAG.

In an embodiment, the first polynucleotide molecule can comprise one or more polynucleotides encoding a protein tag and the second polynucleotide molecule can comprise one or more polynucleotides encoding a protein tag.

Polynucleotide molecules described herein can include one or more protein tags. For example, a polynucleotide can comprise 1, 2, 3, 4, or more protein tags of any type. The protein tag sequence can occur anywhere in the molecule. For example, a protein tag sequence can be incorporated at the 5′ end of a polynucleotide molecule, at the 3′ end of the molecule, or both at the 5′ and at the 3′ end of the molecule.

Cas Protein Split Prime Editor

An embodiment provides a prime editor comprising: a first polynucleotide molecule encoding a reverse transcriptase, an N-terminal fragment of Cas protein, and an N-terminal fragment of a dimerization protein; and a second polynucleotide molecule encoding a C-terminal fragment of a dimerization protein and a C-terminal fragment of Cas protein. The N-terminal fragment of the Cas protein and the C-terminal fragment of the Cas protein can form a full-length Cas protein when combined.

In an embodiment, a N-terminal fragment of a dimerization protein can be the N-terminal fragment of an intein; and a C-terminal fragment of a dimerization protein can be the C-terminal fragment of an intein.

In an embodiment, a first polynucleotide molecule can comprise one or more nuclear localization signals and the second polynucleotide molecule can comprise one or more nuclear localization signals.

In an embodiment, a first polynucleotide molecule can comprise two NLS sequences, a reverse transcriptase, an N-terminal fragment of Cas nickase, and an N-terminal fragment of an intein transcriptase. The Cas protein can be a Cas nickase, a Cas9 nickase, a dead Cas protein, a dead Cas9, or an active Cas protein. For example, a first polynucleotide molecule can comprise a first NLS sequence, a reverse transcriptase, a second NLS sequence, a N-terminal fragment of a Cas9 nickase, and a N-terminal fragment of an intein. In an embodiment, a N-terminal fragment of an intein can comprise SEQ ID NO:3.

In another embodiment, a second polynucleotide molecule can comprise two NLS sequences, a C-terminal fragment of Cas nickase, and a C-terminal fragment of an intein. For example, a second polynucleotide molecule can comprise a first NLS sequence, a C-terminal fragment of an intein, a C-terminal fragment of a Cas9 nickase, and a second NLS sequence. In an embodiment, a C-terminal fragment of an intein can comprise SEQ ID NO:7.

In an embodiment, Cas nickase can be split into a N-terminal fragment and a C-terminal fragment.

In an embodiment, the Cas9 nickase can be split into a N-terminal fragment and a C-terminal fragment at a split point.

In an embodiment, the split point can be localized at any amino acid between position 564 and 584 (e.g. 564, 565, 566, 567, 568, 569, 570, 571, 572, 573, 574, 575, 576, 577, 578, 579, 580, 581, 582, 583, 584), and the N-terminal fragment of Cas9 nickase can comprise nucleotides from position 1 of a Cas9 nickase to the split point and the C-terminal fragment of Cas9 nickase can comprise nucleotides from the split point to position 1371 of a Cas9 nickase. In an embodiment, a first polynucleotide molecule can comprise a reverse transcriptase, nucleotides 1-574 of a Cas9 nickase and an N-terminal fragment of an intein. A second polynucleotide molecule can comprise a C-terminal fragment of an intein and nucleotides 575-1371 of a Cas9 nickase. For example, a first polynucleotide molecule can comprise SEQ ID NO:5, and a second polynucleotide molecule can comprise SEQ ID NO:6. In another embodiment, the split point can be localized at any amino acid between position 249 and 269 (e.g., 249, 250, 251, 252, 253, 254, 255, 256, 257, 258, 259, 260, 261, 262, 263, 264, 265, 266, 267, 268, 269), and the N-terminal fragment of Cas9 nickase can comprise nucleotides from position 1 of a Cas9 nickase to the split point and the C-terminal fragment of Cas9 nickase can comprise nucleotides from the split point to position 1371 of a Cas9 nickase. In an embodiment, a first polynucleotide molecule can comprise an N-terminal fragment of an intein, nucleotides 1-259 of a Cas9 nickase, and a reverse transcriptase. A second polynucleotide molecule comprising nucleotides 260-1371 of a Cas9 nickase and a C-terminal fragment of an intein.

In an embodiment, the split point can be localized at any amino acid between position 265 and 285 (e.g., 265, 266, 267, 268, 269, 270, 271, 272, 273, 274, 275, 276, 277, 278, 279, 280, 281, 282, 283, 284, 285), and the N-terminal fragment of Cas9 nickase can comprise nucleotides from position 1 of a Cas9 nickase to the split point and the C-terminal fragment of Cas9 nickase can comprise nucleotides from the split point to position 1371 of a Cas9 nickase. In an embodiment, a first polynucleotide molecule can comprise a reverse transcriptase, nucleotides 1-275 of a Cas9 nickase and an N-terminal fragment of an intein. A second polynucleotide molecule can comprise nucleotides 276-1371 of a Cas9 nickase and a C-terminal fragment of an intein.

Another embodiment provides a prime editor comprising: a first polynucleotide molecule encoding a N-terminal fragment of a Cas protein, and a N-terminal fragment of a dimerization protein; and a second polynucleotide molecule encoding a C-terminal fragment of a dimerization protein, a C-terminal fragment of Cas protein, and a reverse transcriptase.

In an embodiment, a N-terminal fragment of a dimerization protein can be the N-terminal fragment of an intein; and a C-terminal fragment of a dimerization protein can be the C-terminal fragment of an intein.

In an embodiment, a first polynucleotide molecule can comprise one or more nuclear localization signals and the second polynucleotide molecule can comprise one or more nuclear localization signals.

In an embodiment, a first polynucleotide molecule can comprise two NLS sequences, an N-terminal fragment of Cas nickase, and an N-terminal fragment of an intein transcriptase. The Cas protein can be a Cas nickase, a Cas9 nickase, a dead Cas protein, a dead Cas9, or an active Cas protein. For example, a first polynucleotide molecule can comprise a first NLS sequence, a N-terminal fragment of a Cas9 nickase, a second NLS sequence, and a N-terminal fragment of an intein. In an embodiment, a N-terminal fragment of an intein can comprise SEQ ID NO:3.

In another embodiment, a second polynucleotide molecule can comprise three NLS sequences, a C-terminal fragment of an intein a C-terminal fragment of Cas nickase, and a reverse transcriptase. For example, a second polynucleotide molecule can comprise a first NLS sequence, a C-terminal fragment of an intein, a second NLS sequence, a C-terminal fragment of a Cas9 nickase, a third NLS sequence and a reverse transcriptase. In an embodiment, a C-terminal fragment of an intein can comprise SEQ ID NO:7.

In an embodiment, the Cas9 nickase can be split into a N-terminal fragment and a C-terminal fragment at a split point.

In an embodiment, the split point can be localized at any amino acid between position 703 and 723, and the N-terminal fragment of Cas9 nickase can comprise nucleotides from position 1-713 of the Cas9 nickase to the split point and the C-terminal fragment of Cas9 nickase can comprise nucleotides from the split point to position 714-1371 of the Cas9 nickase. In an embodiment, a first polynucleotide molecule comprising an N-terminal fragment of an intein, nucleotides 1-713 of a Cas9 nickase and a reverse transcriptase. A second polynucleotide molecule can comprise nucleotides 714-1371 of a Cas9 nickase and a C-terminal fragment of an intein. For example, a first polynucleotide molecule can comprise SEQ ID NO:9, and a second polynucleotide molecule can comprise SEQ ID NO:10.

In an embodiment, the split point can be localized at any amino acid between position 935 and 965 (e.g., 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965), and the N-terminal fragment of Cas9 nickase can comprise nucleotides from position 1-945 of the Cas9 nickase to the split point and the C-terminal fragment of Cas9 nickase can comprise nucleotides from the split point to position 946-1371 of the Cas9 nickase. Cas9 nickase can be split into a N-terminal fragment and a C-terminal fragment at amino acid 945. In an embodiment, a first polynucleotide molecule can comprise nucleotides 1-945 of a Cas9 nickase and an N-terminal fragment of an intein. A second polynucleotide molecule can comprise a C-terminal fragment of an intein nucleotides 946-1371 of a Cas9 nickase and a reverse transcriptase. For example, a first polynucleotide molecule can comprise SEQ ID NO:11, and a second polynucleotide molecule can comprise SEQ ID NO:12.

In another embodiment, the split point can be localized at any amino acid between position 1044 and 1064 (e.g., 1044, 1045, 1046, 1047, 1048, 1049, 1050, 1051, 1052, 1053, 1054, 1055, 1056, 1057, 1058, 1059, 1060, 1061, 1062, 1063, 1064) and, the N-terminal fragment of Cas9 nickase can comprise nucleotides from position 1-1054 of the Cas9 nickase to the split point and the C-terminal fragment of Cas9 nickase can comprise nucleotides from the split point to position 1055-1371 of the Cas9 nickase. Cas9 nickase can be split into a N-terminal fragment and a C-terminal fragment at amino acid 1054. In an embodiment, a first polynucleotide molecule can comprise nucleotides 1-1054 of a Cas9 nickase and an N-terminal fragment of an intein. A second polynucleotide molecule can comprise a C-terminal fragment of an intein nucleotides 1055-1371 of a Cas9 nickase and a reverse transcriptase. For example, a first polynucleotide molecule can comprise SEQ ID NO:13, and a second polynucleotide molecule can comprise SEQ ID NO:14.

In an embodiment, the split point can localized at any amino acid between position 1105 and 1125 (e.g., 1105, 1106, 1107, 1108, 1109, 1110, 1111, 1112, 1113, 1114, 1115, 1116, 1117, 1118, 1119, 1120, 1121, 1122, 1123, 1124, 1125), and the N-terminal fragment of Cas9 nickase can comprise nucleotides from position 1-1115 of the Cas9 nickase to the split point and the C-terminal fragment of Cas9 nickase can comprise nucleotides from the split point to position1116-1371 of the Cas9 nickase.Cas9 nickase can be split into a N-terminal fragment and a C-terminal fragment at amino acid 1115. In an embodiment, a first polynucleotide molecule can comprise nucleotides 1-1115 of a Cas9 nickase and an N-terminal fragment of an intein. A second polynucleotide molecule can comprise a C-terminal fragment of an intein nucleotides 1116-1371 of a Cas9 nickase and a reverse transcriptase. For example, a first polynucleotide molecule can comprise SEQ ID NO:16, and a second polynucleotide molecule can comprise SEQ ID NO:16.

Therefore, a Cas9 nickase can be split into a N-terminal fragment and a C-terminal fragment at many different positions.

The amino acid positions described herein to split Cas9 (i.e., amino acid positions 259, 275, 574, 713, 945, 1054, and 1115) located within a short β-strand; therefore, splitting Cas9 at any of the amino acid within the β-strand is expected to be as efficient as splitting Cas9 at the exact amino acid. β-strand, or β-pleated sheet are common motifs of regular secondary structure in proteins. Beta sheets consist of beta strands (also β-strand) connected laterally by at least two or three backbone hydrogen bonds, forming a generally twisted, pleated sheet. A β-strand is a stretch of polypeptide chain typically 3 to 10 amino acids long with backbone in an extended conformation. Accordingly, a split of a Cas9 can be located 1, 2, 3, 4, 5, 6, 7, 8, 9, or 10 amino acid upstream or downstream of the split position. Additionally, target sites for splitting the Cas protein can be used. For example, any surface exposed loops, any other site that does not interfere with native Cas9 activity, or any site where reconstituted split Cas9 retains full or partial activity of full-length Cas9 (such as position 111 or 112) can be used as a split point.

For example, a Cas9 can be split at position 259, or at position 258, 257, 256, 255, 254, 253, 252, 251, 250, or 249 (i.e., up to 10 amino acid upstream of position 259), or at position 260, 262, 262, 263, 264, 265, 266, 267, 268, or 269 (up to 10 amino acid downstream of position 259).

A Cas9 can be split at position 275, or at position 274, 273, 272, 271, 270, 269, 268, 267, 266, or 265 (i.e., up to 10 amino acid upstream of position 275), or at position 276, 277, 278, 279, 280, 281, 282, 283, 284, or 285 (up to 10 amino acid downstream of position 275).

A Cas9 can be split at position 574, or at position 573, 572, 571, 570, 569, 568, 567, 566, 565, or 564 (i.e., up to 10 amino acid upstream of position 574), or at position 575, 576, 577, 578, 579, 580, 581, 582, 583, or 584 (up to 10 amino acid downstream of position 574).

A Cas9 can be split at position 713, or at position 712, 711, 710, 709, 708, 707, 706, 705, 704, or 703 (i.e., up to 10 amino acid upstream of position 713), or at position 714, 715, 716, 717, 718, 719, 720, 721, 722, or 723 (up to 10 amino acid downstream of position 713).

A Cas9 can be split at position 945, or at position 944, 943, 942, 941, 940, 939, 938, 937, 936, or 935 (i.e., up to 10 amino acid upstream of position 945), or at position 946, 947, 948, 949, 950, 951, 952, 953, 954, or 955 (up to 10 amino acid downstream of position 945).

A Cas9 can be split at position 1054, or at position 1053, 1052, 1051, 1050, 1049, 1048, 1047, 1046, 1045, or 1044 (i.e., up to 10 amino acid upstream of position 1054), or at position 1055, 1056, 1057, 1058, 1059, 1060, 1061, 1062, 1063, or 1064 (up to 10 amino acid downstream of position 1054).

A Cas9 can be split at position 1115, or at position 1114, 1113, 1112, 1111, 1110, 1109, 1108, 1107, 1106, or 1105 (i.e., up to 10 amino acid upstream of position 1115), or at position 1116, 1117, 1118, 1119, 1120, 1121, 1122, 1123, 1124, or 1125 (up to 10 amino acid downstream of position 1115).

System for Prime Editing

An embodiment provides a system for prime editing comprising: a first vector comprising a first polynucleotide molecule encoding a Cas protein and an N-terminal fragment of a dimerization protein; and a second vector comprising a second polynucleotide molecule encoding a C-terminal fragment of a dimerization protein and a reverse transcriptase.

Another embodiment provides a system for prime editing comprising: a first vector comprising a first polynucleotide molecule encoding a reverse transcriptase, an N-terminal fragment of Cas protein, and an N-terminal fragment of a dimerization protein; and a second vector comprising a second polynucleotide molecule encoding a C-terminal fragment of a dimerization protein and a C-terminal fragment of Cas protein.

An embodiment provides a system for prime editing comprising: a first vector comprising a first polynucleotide molecule encoding a reverse transcriptase, an N-terminal fragment of Cas protein, and an N-terminal fragment of a dimerization protein; and a second vector comprising a second polynucleotide molecule encoding a C-terminal fragment of a dimerization protein and a C-terminal fragment of Cas protein.

Another embodiment provides a system for prime editing comprising: a first vector comprising a first polynucleotide molecule encoding a N-terminal fragment of a Cas protein, and a N-terminal fragment of a dimerization protein; and a second vector comprising a second polynucleotide molecule encoding a C-terminal fragment of a dimerization protein, a C-terminal fragment of Cas protein, and a reverse transcriptase.

In an embodiment, a system for prime editing can comprise a first vector comprising a first polynucleotide molecule encoding a Cas9 nickase and an N-terminal fragment of an intein; and a second vector comprising a second polynucleotide molecule encoding a C-terminal fragment of an intein and a reverse transcriptase.

In another embodiment, a system for prime editing can comprise a first vector comprising a first polynucleotide molecule encoding a reverse transcriptase, an N-terminal fragment of Cas9 nickase, and an N-terminal fragment of an intein; a second vector comprising a second polynucleotide molecule encoding a C-terminal fragment of an intein and a C-terminal fragment of Cas9 nickase.

Polynucleotides can be delivered to cells (e.g., a plurality of different cells or cell types including target cells or cell types and/or non-target cell types) in a vector (e.g., an expression vector). Examples of vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs); episomal vectors; transposons (e.g., PiggyBac); and (b) viral vectors such as retroviral vectors, lentiviral vectors, adenoviral vectors, and AAV vectors. Viral vectors have several advantages for delivery of nucleic acids, including high infectivity and/or tropism for certain target cells or tissues. In some cases, a viral vector can be used to deliver a polynucleotides described herein.

In an embodiment the vector is an AAV vector. The term “AAV” is an abbreviation for adeno-associated virus, and can be used to refer to the virus itself or a derivative thereof. The term covers all serotypes, subtypes, and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV1, AAV2, AAV3, AAV4, AAV5, AAV6, AAV7, AAV8, AAV9, AAV10, AAV11, AAV12, AAVDJ, rhlO, derivatives and hybrids thereof, avian AAV, bovine AAV, canine AAV, equine AAV, primate AAV, non-primate AAV, and ovine AAV. Additionally, any engineered or variant derived from ancestral AAV sequence reconstruction can be used as a vector. The genomic sequences of various serotypes of AAV, as well as the sequences of the native terminal repeats (TRs), Rep proteins, and capsid subunits are known in the art. Such sequences may be found in the literature or in public databases such as GenBank. An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two, AAV inverted terminal repeat sequences (ITRs). The term rAAV vector encompasses both rAAV vector particles and rAAV vector plasmids. An rAAV vector may either be single-stranded (ssAAV) or self-complementary (scAAV). An “AAV virus” or “AAV viral particle” or “rAAV vector particle” refers to a viral particle composed of at least one AAV capsid protein and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

Techniques contemplated herein for gene therapy of somatic cells include delivery via a viral vector (e.g., retroviral, adenoviral, AAV, helper-dependent adenoviral systems, hybrid adenoviral systems, herpes simplex, pox virus, lentivirus, and Epstein-Barr virus), and non-viral systems, such as physical systems (naked DNA, DNA bombardment, electroporation, hydrodynamic, ultrasound, and magnetofection), and chemical systems (cationic lipids, different cationic polymers, and lipid polymers).

In some aspects, instead of delivering the full-sized protein or the gene encoding it, the prime editors described herein can be produced and purified in vitro, and delivered as ‘proteins+guide RNAs’, by the same physical and chemical methods used for DNA delivery.

The cloning capacity of vectors or viral expression vectors is a particular challenge for expression of large transgenes. For example, AAV vectors typically have a packaging capacity of ˜4.8 kb, lentiviruses typically have a capacity of ˜8 kb, adenoviruses typically have a capacity of ˜7.5 kb and alphaviruses typically have a capacity of ˜7.5 kb. Some viruses can have larger packaging capacities, for example herpesvirus can have a capacity of >30 kb and vaccinia a capacity of ˜25 kb. Advantages of using AAV for gene therapy include low pathogenicity, very low frequency of integration into the host genome, and the ability to infect dividing and non-dividing cells.

Gene delivery vectors, including viral gene therapy vectors, can have the ability to be reproducible and stably propagated and purified to high titers; to mediate targeted delivery (e.g., to deliver the transgene specifically to a tissue or organ of interest without widespread vector dissemination elsewhere or off-target delivery); and to mediate gene delivery and/or transgene expression without inducing harmful side effects or off-target effects.

Methods of Use of the Prime Editors.

An embodiment provides methods of editing genomic DNA in a cell comprising contacting the cell with a system for prime editing.

The system can further comprise one or more pegRNA molecules, one or more sgRNA molecules, or a combination of one or more pegRNA molecules and one or more sgRNA molecule.

As used herein, “prime editing guide RNA,” and “pegRNA” can be used interchangeably and refer to a single RNA species that is capable of identifying a target nucleotide sequence to be edited and encodes the genetic information to be incorporated at the targeted sequence. pegRNA sequences are transcribed from double-stranded DNA sequences inside the cell. A pegRNA recognizes a target DNA region of interest and directs an RNA-guided DNA endonuclease there for editing. A pegRNA has at least three regions. First, a spacer sequence (or protospacer), which is a nucleotide sequence complementary to the target nucleic acid, second a structure allowing the hybridization of the pegRNA and Cas9 (such as a loop), and which serves as a binding scaffold for the RNA-guided DNA endonuclease), and third a sequence that primes the reverse transcriptase and provides the template for introducing targeted modifications (i.e., primer binding site (PBS) and RT template). The spacer RNA, the sgRNA scaffold (loop) and the PBS/RT template can exist as one molecule or as two separate molecules. pegRNA refer to a single molecule comprising at least a spacer RNA region, a loop, and an RT template region or two separate molecules wherein the first comprises the spacer RNA region and the second comprises rest of the pegRNA. The spacer RNA region of the pegRNA is a customizable component that enables specificity in every prime editing reaction. The PBS/RT template is also customizable. pegRNA used in the systems and methods described herein can be short, single-stranded polynucleotide molecules from about 20 nucleotides to about 300 nucleotides in length. The spacer sequence (targeting sequence) that hybridizes to a complementary region of the target DNA of interest can be about 14, 15, 16, 17, 18, 19, 20, 25, 30, 35 or more nucleotides in length. A PBS/RT template capable of directing RNA-guided DNA endonuclease mediated substitution of, insertion at, or deletion of target sequence can be about 10, 11, 12, 13, 14, 15, 16, 17, 18, 19, 20, 30, 40, 50 or more nucleotides in length. A PBS/RT template capable of directing RNA-guided DNA endonuclease mediated substitution of, insertion at, or deletion of target sequence can be about 50, 40, 30, 20, 19, 18, 17, 16, 15, 14, 13, 12, 11 or less nucleotides in length. pegRNAs can be synthetically generated or by making the pegRNA in vivo or in vitro, starting from a DNA template. A pegRNA can target a regulatory element (e.g., a promoter, enhancer, or other regulatory element) in the target genome, or any DNA in general. A pegRNA can also target a protein coding sequence in the target genome.

A pegRNA can be used alone (PE2), in combination with a sgRNA (PE3 or PE3b) or a modified pegRNA developed to improve editing of some targets. These modifications can be, for example, mutations in the loop, addition of additional loops or incorporation of aptamers to recruit additional reverse transcriptase subunits, or any other enzyme that could improve editing outcomes, to the target site.

The one or more pegRNA molecules can comprise one or more loops, one or more base modifications, or a combination of one or more loops and one or more base modifications to enhance prime editing activity. Any additional modifications such as to the linker between the gRNA loop and the PBS/RT sequence can be incorporated to improve the efficacy of the pegRNA.

The one or more pegRNA molecules can comprise SEQ ID NOs:17, 18, 19, 20, or 21.

In an embodiment, prime editing genomic DNA can avoid generating a double-stranded break. Editing genomic DNA can induce an insertion, deletion, transversion point mutation, or transition point mutation. A first vector and a second vector can be AAV vectors.

In an embodiment, upon contacting of a cell (i.e., infection of a cell) with a first vector comprising a first polynucleotide molecule encoding a Cas9 nickase and an N-terminal fragment of an intein; and a second vector comprising a second polynucleotide molecule encoding a C-terminal fragment of an intein and a reverse transcriptase; a cell can express a first polypeptide molecule comprising a Cas9 nickase fused to an N-terminal fragment of an intein, and a second polypeptide molecule comprising a C-terminal fragment of an intein fused to a reverse transcriptase. An N-terminal fragment of an intein and a C-terminal fragment of an intein can splice to generate a mature intein protein, and excise itself and generate a fusion protein between a Cas9 nickase and a reverse transcriptase. A Cas9 nickase:reverse transcriptase fusion protein can then prime edit genomic DNA in the cell.

In another embodiment, upon contacting of a cell (i.e., infection of a cell) with a first vector comprising a first polynucleotide molecule encoding a reverse transcriptase, an N-terminal fragment of Cas9 nickase, and an N-terminal fragment of an intein; and a second vector comprising a second polynucleotide molecule encoding a C-terminal fragment of an intein and a C-terminal fragment of Cas9 nickase; a cell can express a first polypeptide molecule comprising a reverse transcriptase fused N-terminal fragment of Cas9 nickase fused to an N-terminal fragment of an intein, and a second polypeptide molecule comprising a C-terminal fragment of an intein fused to a C-terminal fragment of Cas9 nickase. An N-terminal fragment of an intein and a C-terminal fragment of an intein can splice to generate a mature intein protein, and excise itself and generate a fusion protein between a reverse transcriptase and an N-terminal fragment of Cas9 nickase and a C-terminal fragment of a Cas9 nickase (i.e., a reverse transcriptase and a full-length Cas9 nickase). A Cas9 nickase:reverse transcriptase fusion protein can then, directed by the pegRNA, prime edit genomic DNA in the cell.

In an embodiment, the methods can further comprise contacting the cell with one or more small molecules. Small molecules such as valproic acid can be used to modify the chromatin and modulate editing efficiency.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined below to provide additional guidance to the practitioner regarding the description of the compositions and methods. As used in the description herein and throughout the claims that follow, the meaning of “a”, “an”, and “the” includes plural reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

Any single term, single element, single phrase, group of terms, group of phrases, or group of elements described herein can be each be specifically excluded from the claims.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### EXAMPLES

### Example 1. Development of Split Prime Editors

Despite their considerable potential for treating human diseases, the implementation of prime editors for in vivo gene correction is limited by the fact that their size exceeds the carrying capacity of a single AAV vector. To overcome the packaging limitations associated with delivering prime editors, prime editors that can be split in two different domains, each of which being fused with an intein-based trans-splicing system, were developed. Following delivery to cells and expression, the inteins bound to each other and excised themselves out while creating a peptide bond between the two prime editor domains that reconstitutes the full-length protein.

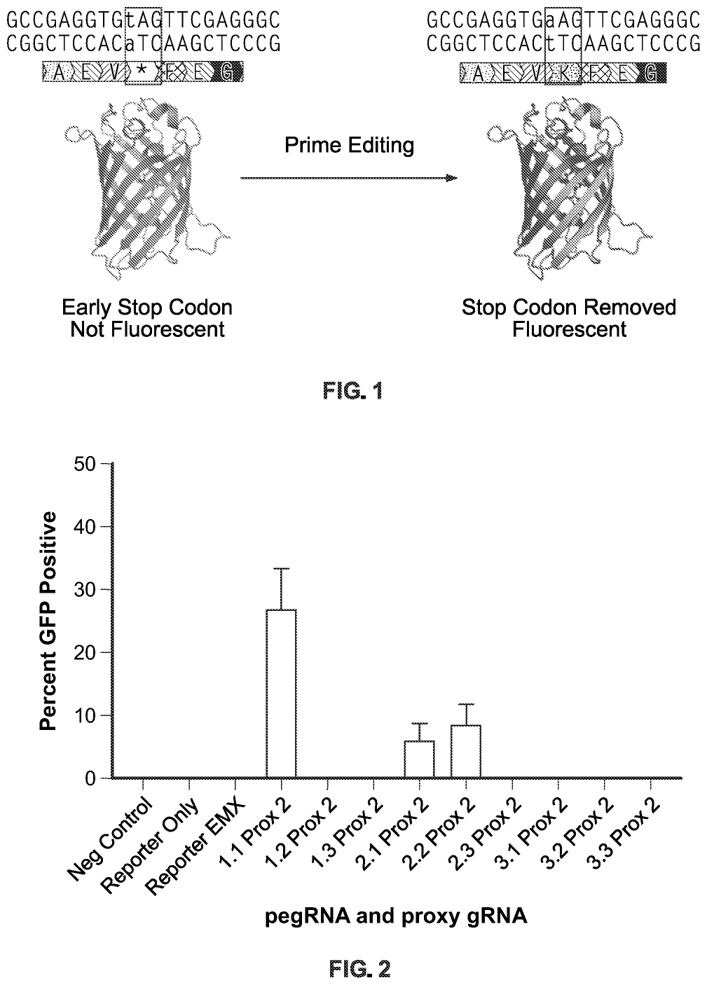

As illustrated in FIG. 1, to enable rapid testing of prime editors, an early stop codon was introduced in a GFP transgene which resulted in a truncated GFP that is not fluorescent. Introduction of a T>A mutation, reverts the TAG stop codon to AAG, which restores the integrity of the wild-type GFP and recovers the normal fluorescence.

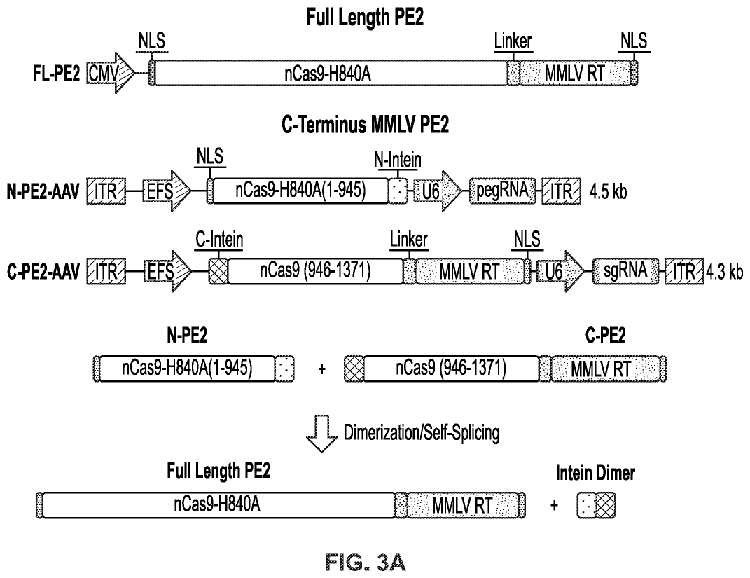

Multiple pegRNAs and proxy sgRNAs targeting the stop codon in the GFP reporter were designed and the different combinations were tested by transfecting the prime editors in combination with the guides and the reporter plasmid in HEK293T cells. GFP expression was then measured using FACS 48 hours after transfection. As illustrated in FIG. 2, certain combinations, such as pegRNA 1.1 in combination with proxy sgRNA 2, were found most effective and can accomplish a modification rate of nearly 27% in this system.

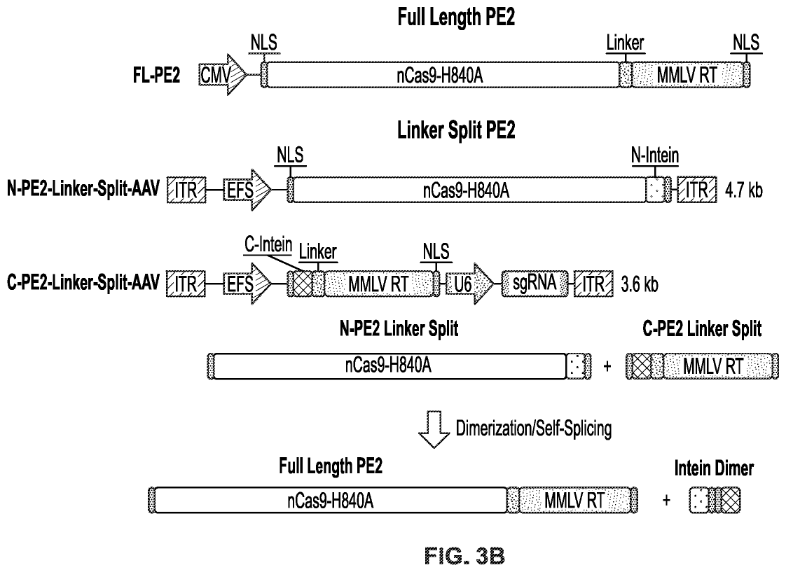

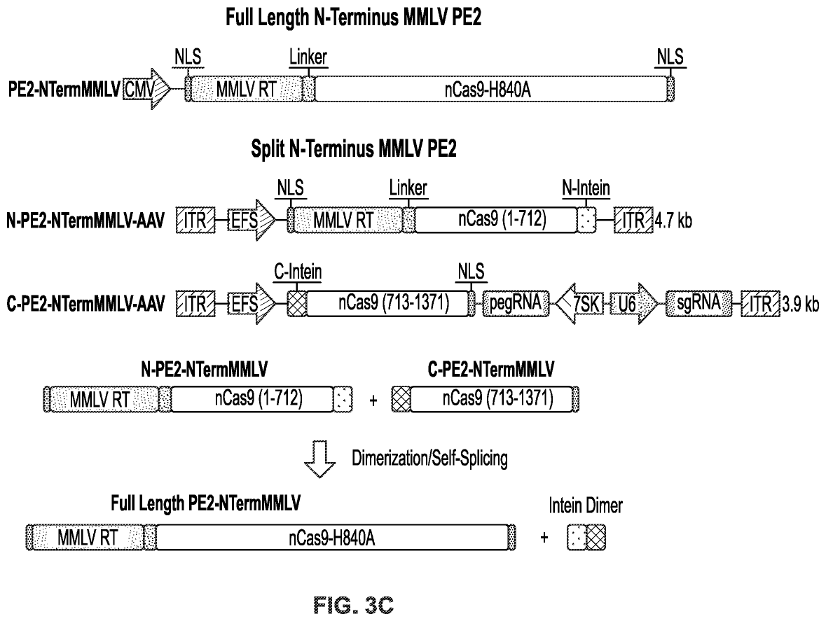

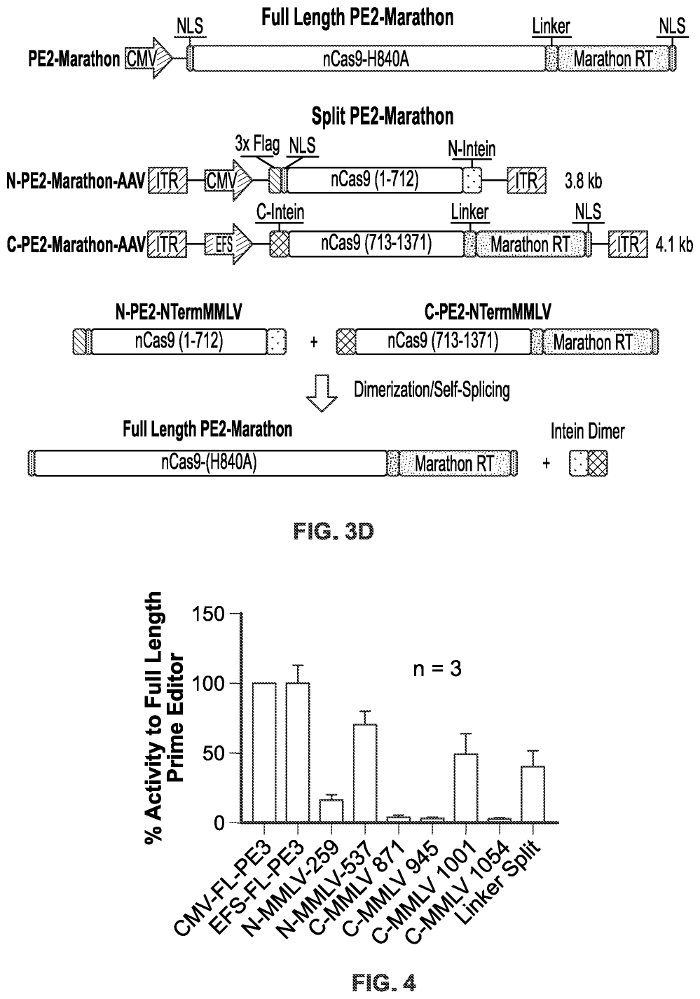

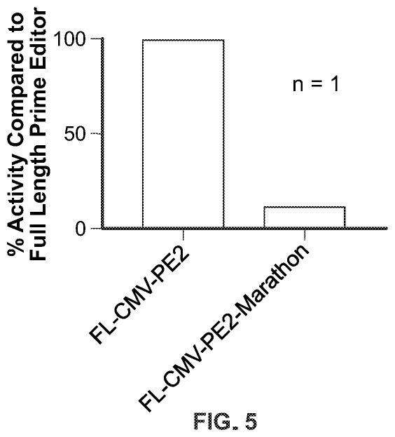

Using the combination of pegRNA and proxyRNA, the efficiency of several split forms of the prime editor were then compared, where the split point was located at the linker between Cas9 and MMLV. As illustrated in FIGS. 3A and 3B, the split editor architecture that was most active comprised one plasmid that contained the full-length Cas9 fused with an N-terminus NPU intein and a separate plasmid that contained the reverse transcriptase fused with the C-terminus plasmid. The activity of this variant was 62% the activity of the wild type prime editor.

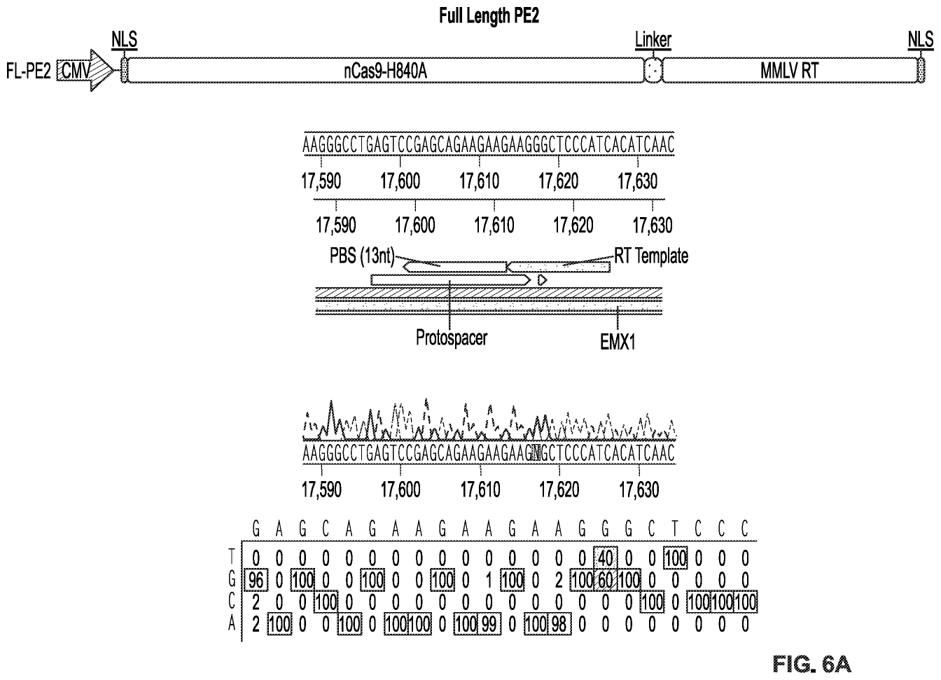

To validate those results, the EMX1 native locus was targeted in genomic DNA using the wild type prime editor and, separately, a split prime editor. The modification rate in genomic DNA was analyzed by Sanger sequencing and, as shown in FIGS. 4A and 4B, while the wild type editor introduced a targeted modification in 40% of the alleles, the split version modified 32% of the alleles.

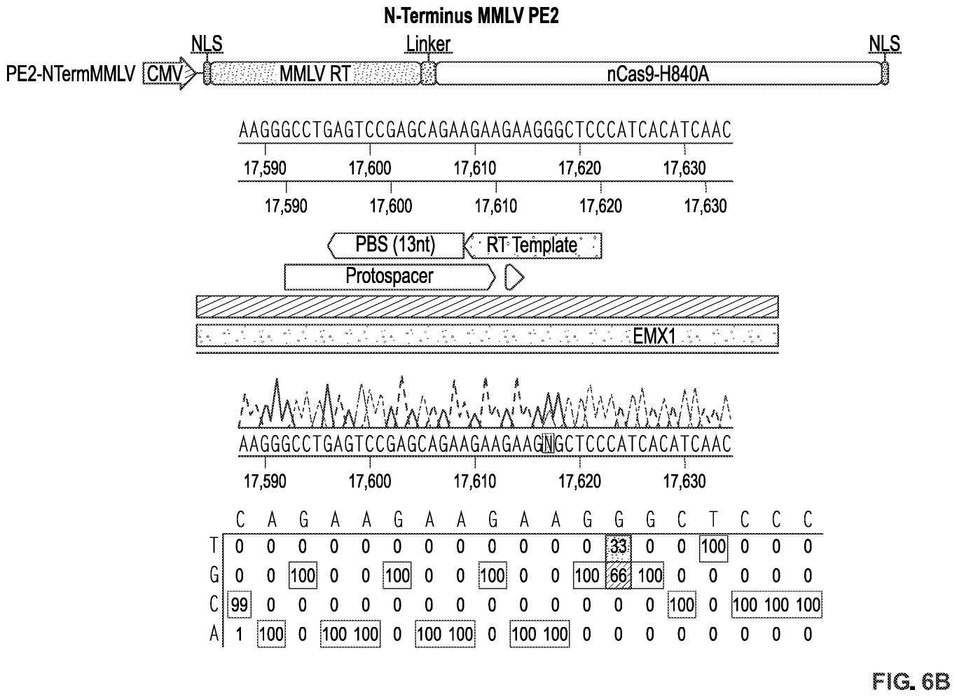

The efficiency of WT prime editors, in which the reverse transcriptase is at the C-terminus, was compared with prime editors in which the reverse transcriptase is at the N-terminus. The results (see FIG. 5) demonstrated that the N-MMLV prime editor was active.

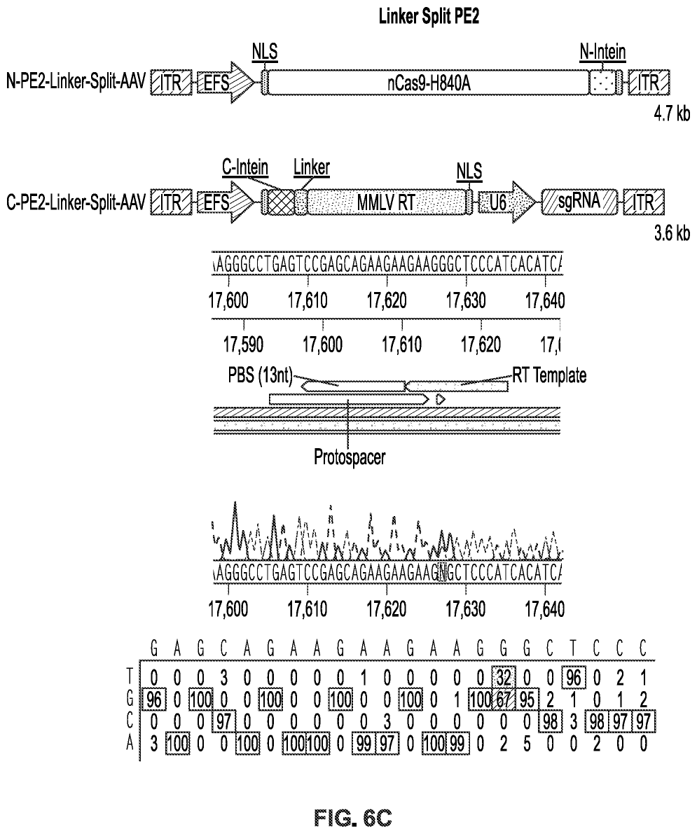

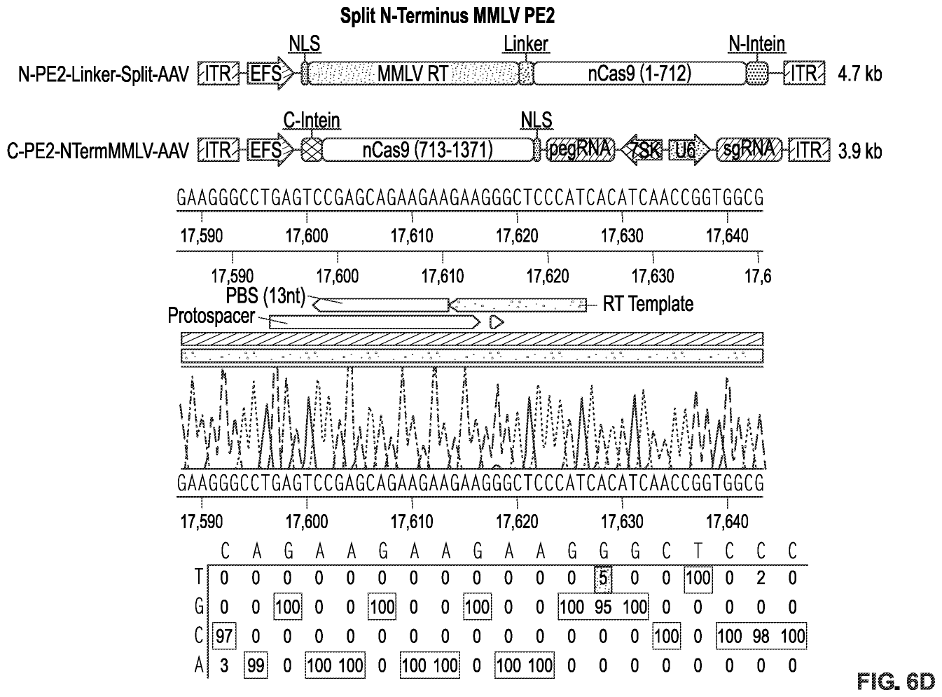

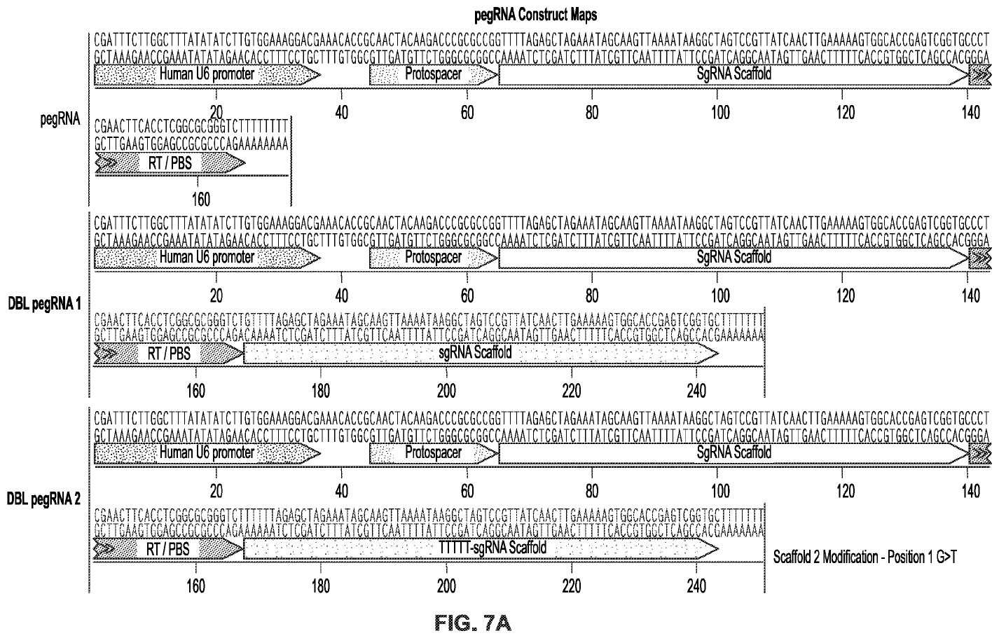

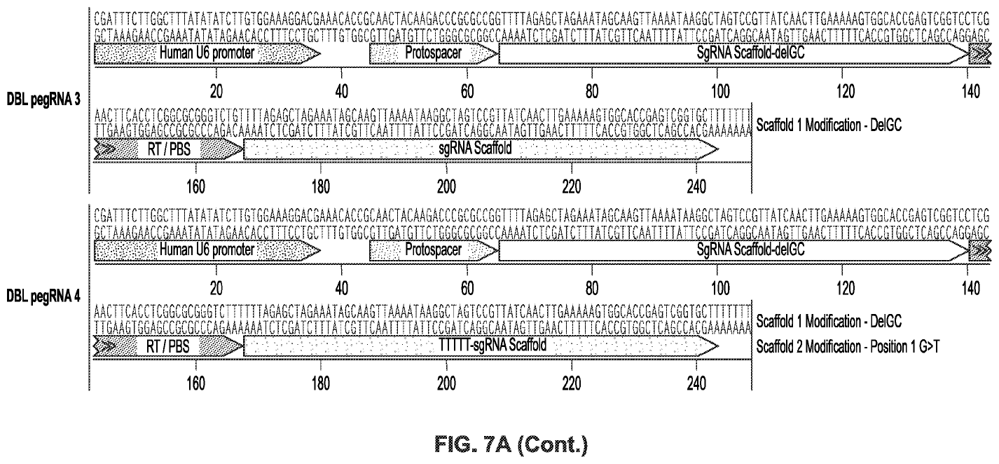

The efficiency of the full-length prime editor with the MMLV at the N-terminus and a corresponding split version at amino acid 575 in Cas9 were also compared (see FIG. 6A). As shown in FIG. 6B, these results demonstrated that the split N-MMLV prime editor was active.

The activity of the full-length prime editor with the MMLV at the N-terminus with the corresponding split version at the EMX1 native locus instead of the reporter system was analyzed. As illustrated in FIGS. 7A and 7B, this split prime editor was also active in native genomic DNA.

The efficiency of the full-length prime editor with the Marathon reverse transcriptase, and a split at amino acid 713, 945, 1054 or 1115 in Cas9 were also compared. As illustrated in FIG. 8, all the split primer editors tested were active, and provided a relative fluorescence that was greater than the relative fluorescence observed with the control.

SEQUENCES:

SEQ ID NO: 1 (Amino acid sequence of the N-terminal fragment of the
split prime editor at the linker)
MKRTADGSEFESPKKKRKVDKKYSIGLDIGTNSVGWAVITDEYKVPSKKFKV
LGNTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYD
VDAIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLI
REVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNSDKLIARK
KDWDPKKYGGFDSPTVAYSVLVVAKVEKGKSKKLKSVKELLGITIMERSSFEKNPID
FLEAKGYKEVKKDLIIKLPKYSLFELENGRKRMLASAGELQKGNELALPSKYVNFLYL
ASHYEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQSITGLY
ETRIDLSQLGGDSGGSSGCLSYETEILTVEYGLLPIGKIVEKRIECTVYSVDNNGNIY
TQPVAQWHDRGEQEVFEYCLEDGSLIRATKDHKFMTVDGQMLPIDEIFERELDLMR
VDNLPNSGGSKRTADGSEFEPKKKRKV
SEQ ID NO: 2 Amino acid sequence of the C-terminus fragment of the
split prime editor at the linker
MKRTADGSEFESPKKKRKVIKIATRKYLGKQNVYDIGVERDHNFALKNGFIA
SNGRAGGSSGSETPGTSESATPESSGGSSGGSSTLNIEDEYRLHETSKEPDVSLG
STWLSDFPQAWAETGGMGLAVRQAPLIIPLKATSTPVSIKQYPMSQEARLGIKPHIQ
RLLDQGILVPCQSPWNTPLLPVKKPGTNDYRPVQDLREVNKRVEDIHPTVPNPYNL
LSGLPPSHQWYTVLDLKDAFFCLRLHPTSQPLFAFEWRDPEMGISGQLTWTRLPQ
GFKNSPTLFNEALHRDLADFRIQHPDLILLQYVDDLLLAATSELDCQQGTRALLQTL
GNLGYRASAKKAQICQKQVKYLGYLLKEGQRWLTEARKETVMGQPTPKTPRQLRE
FLGKAGFCRLFIPGFAEMAAPLYPLTKPGTLFNWGPDQQKAYQEIKQALLTAPALGL
PDLTKPFELFVDEKQGYAKGVLTQKLGPWRRPVAYLSKKLDPVAAGWPPCLRMVA
AIAVLTKDAGKLTMGQPLVILAPHAVEALVKQPPDRWLSNARMTHYQALLLDTDRV
QFGPVVALNPATLLPLPEEGLQHNCLDILAEAHGTRPDLTDQPLPDADHTWYTDGS
SLLQEGQRKAGAAVTTETEVIWAKALPAGTSAQRAELIALTQALKMAEGKKLNVYT
DSRYAFATAHIHGEIYRRRGWLTSEGKEIKNKDEILALLKALFLPKRLSIIHCPGHQK
GHSAEARGNRMADQAARKAAITETPDTSTLLIENSSPSGGSKRTADGSEFEPKKKR
KV
SEQ ID NO: 3 sequence of the N-terminal fragment of a split intein (Part
of SEQ ID NO: 1)
CLSYETEILTVEYGLLPIGKIVEKRIECTVYSVDNNGNIYTQPVAQWHDRGEQ
EVFEYCLEDGSLIRATKDHKFMTVDGQMLPIDEIFERELDLMRVDNLPN
SEQ ID NO: 4 (Part of SEQ ID NO: 2).
IKIATRKYLGKQNVYDIGVERDHNFALKNGFIASNGRAGGSSGSETPGTSES
ATPESSGGSSGGSS
SEQ ID NO: 5 Amino acid sequence of the N-terminus fragment of the
Split prime editor with the MMLV at the N-terminus
MGPKKKRKVGGSSTLNIEDEYRLHETSKEPDVSLGSTWLSDFPQAWAETG
GMGLAVRQAPLIIPLKATSTPVSIKQYPMSQEARLGIKPHIQRLLDQGILVPCQSPWN
TPLLPVKKPGTNDYRPVQDLREVNKRVEDIHPTVPNPYNLLSGLPPSHQWYTVLDL
KDAFFCLRLHPTSQPLFAFEWRDPEMGISGQLTWTRLPQGFKNSPTLFNEALHRDL
ADFRIQHPDLILLQYVDDLLLAATSELDCQQGTRALLQTLGNLGYRASAKKAQICQK
QVKYLGYLLKEGQRWLTEARKETVMGQPTPKTPRQLREFLGKAGFCRLFIPGFAE
MAAPLYPLTKPGTLFNWGPDQQKAYQEIKQALLTAPALGLPDLTKPFELFVDEKQG
YAKGVLTQKLGPWRRPVAYLSKKLDPVAAGWPPCLRMVAAIAVLTKDAGKLTMGQ
PLVILAPHAVEALVKQPPDRWLSNARMTHYQALLLDTDRVQFGPVVALNPATLLPL
PEEGLQHNCLDILAEAHGTRPDLTDQPLPDADHTWYTDGSSLLQEGQRKAGAAVT
TETEVIWAKALPAGTSAQRAELIALTQALKMAEGKKLNVYTDSRYAFATAHIHGEIYR
RRGWLTSEGKEIKNKDEILALLKALFLPKRLSIIHCPGHQKGHSAEARGNRMADQAA
RKAAITETPDTSTLLIENSSPSGGSKRTADGSEFEPKKKRKVSGGSSGGSSGSETP
GTSESATPESSGGSSGGSSTLEPGEKPYKCPECGKSFSQSGALTRHQRTHTRDK
KYSIGLDIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPD
NSDVDKLFIQLVQTYNQLFEENPINASGVDAKAILSARLSKSRRLENLIAQLPGEKKN
GLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWM
TRKSEETITPWNFEEVVDKGASAQSFIERMTNFDKNLPNEKVLPKHSLLYEYFTVYN
ELTKVKYVTEGMRKPAFLSGEQKKAIVDLLFKTNRKVTVKQLKEDYFKKIECLSYET
EILTVEYGLLPIGKIVEKRIECTVYSVDNNGNIYTQPVAQWHDRGEQEVFEYCLEDG
SLIRATKDHKFMTVDGQMLPIDEIFERELDLMRVDNLPN
SEQ ID NO: 6 Amino acid sequence of the C-terminus fragment of the
Split prime editor with the MMLV at the N-terminus
MKRTADGSEFESPKKKRKVIKIATRKYLGKQNVYDIGVERDHNFALKNGFIA
SNCFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRK
VLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGST
NLSDIIEKETGKQLVIQESILMLPEEVEEVIGNKPESDILVHTAYDESTDENVMLLTSD
APEYKPWALVIQDSNGENKIKMLSYPYDVPDYAYPYDVPDYAYPYDVPDYASGGS
PKKKRKV
SEQ ID NO: 7 C-terminal fragment of a split intein (Part of SEQ ID NO: 6)
IKIATRKYLGKQNVYDIGVERDHNFALKNGFIASN
SEQ ID NO: 8 Marathon Reverse Transcriptase
NLMEQILSSDNLNRAYLQVVRNKGAEGVDGMKYTELKEHLAKNGETIKGQL
RTRKYKPQPARRVEIPKPDGGVRNLGVPTVTDRFIQQAIAQVLTPIYEEQFHDHSYG
FRPNRCAQQAILTALNIMNDGNDWIVDIDLEKFFDTVNHDKLMTLIGRTIKDGDVISIV
RKYLVSGIMIDDEYEDSIVGTPQGGNLSPLLANIMLNELDKEMEKRGLNFVRYADDC
IIMVGSEMSANRVMRNISRFIEEKLGLKVNMTKSKVDRPSGLKYLGFGFYFDPRAH
QFKAKPHAKSVAKFKKRMKELTCRSWGVSNSYKVEKLNQLIRGWINYFKIGSMKTL
CKELDSRIRYRLRMCIWKQWKTPQNQEKNLVKLGIDRNTARRVAYTGKRIAYVQNK
GAVNVAISNKRLASFGLISMLDYYIEKCVTCEFE
SEQ ID NO: 9 N-terminus fragment of Split 713
MKRTADGSEFESPKKKRKVDKKYSIGLDIGTNSVGWAVITDEYKVPSKKFKV
LGNTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVCLAGDTLITLAD
GRRVPIRELVSQQNFSVWALNPQTYRLERARVSRAFCTGIKPVYRLTTRLGRSIRA
TANHRFLTPQGWKRVDELQPGDYLALPRRIPTAS
SEQ ID NO: 10 C-terminus fragment of Split 713
MAAACPELRQLAQSDVYWDPIVSIEPDGVEEVFDLTVPGPHNFVANDIIAHN
SGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVD
QELDINRLSDYDVDAIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILDSR
MNTKYDENDKLIREVKVITLKSKLVSDFRKDFQFYKVREINNYHHAHDAYLNAVVGT
ALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESI
LPKRNSDKLIARKKDWDPKKYGGFDSPTVAYSVLVVAKVEKGKSKKLKSVKELLGIT
IMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKE
VLDATLIHQSITGLYETRIDLSQLGGDSGGSSGGSSGSETPGTSESATPESSGGSS
GGSSTLNIEDEYRLHETSKEPDVSLGSTWLSDFPQAWAETGGMGLAVRQAPLIIPL
KATSTPVSIKQYPMSQEARLGIKPHIQRLLDQGILVPCQSPWNTPLLPVKKPGTNDY
RPVQDLREVNKRVEDIHPTVPNPYNLLSGLPPSHQWYTVLDLKDAFFCLRLHPTSQ
PLFAFEWRDPEMGISGQLTWTRLPQGFKNSPTLFNEALHRDLADFRIQHPDLILLQY
VDDLLLAATSELDCQQGTRALLQTLGNLGYRASAKKAQICQKQVKYLGYLLKEGQR
WLTEARKETVMGQPTPKTPRQLREFLGKAGFCRLFIPGFAEMAAPLYPLTKPGTLF
NWGPDQQKAYQEIKQALLTAPALGLPDLTKPFELFVDEKQGYAKGVLTQKLGPWR
RPVAYLSKKLDPVAAGWPPCLRMVAAIAVLTKDAGKLTMGQPLVILAPHAVEALVK
QPPDRWLSNARMTHYQALLLDTDRVQFGPVVALNPATLLPLPEEGLQHNCLDILAE
AHGTRPDLTDQPLPDADHTWYTDGSSLLQEGQRKAGAAVTTETEVIWAKALPAGT
SAQRAELIALTQALKMAEGKKLNVYTDSRYAFATAHIHGEIYRRRGWLTSEGKEIKN
KDEILALLKALFLPKRLSIIHCPGHQKGHSAEARGNRMADQAARKAAITETPDTSTLL
IENSSPSGGSKRTADGSEFEPKKKRKV
SEQ ID NO: 11 N-terminus fragment of Split 945
MKRTADGSEFESPKKKRKVDKKYSIGLDIGTNSVGWAVITDEYKVPSKKFKV
LGNTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYD
VDAIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNCLAGDTLITL
ADGRRVPIRELVSQQNFSVWALNPQTYRLERARVSRAFCTGIKPVYRLTTRLGRSI
RATANHRFLTPQGWKRVDELQPGDYLALPRRIPTAS
SEQ ID NO: 12 C-terminus fragment of Split 945
MAAACPELRQLAQSDVYWDPIVSIEPDGVEEVFDLTVPGPHNFVANDIIAHN
TKYDENDKLIREVKVITLKSKLVSDFRKDFQFYKVREINNYHHAHDAYLNAVVGTALI
KKYPKLESEFVYGDYKVYDVRKMIAKSEQEIGKATAKYFFYSNIMNFFKTEITLANGE
IRKRPLIETNGETGEIVWDKGRDFATVRKVLSMPQVNIVKKTEVQTGGFSKESILPK
RNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVLD
ATLIHQSITGLYETRIDLSQLGGDPIAGSKASPKKKRKVGRAGGSSGSETPGTSESA
TPESSGGSSGGSSTLNIEDEYRLHETSKEPDVSLGSTWLSDFPQAWAETGGMGLA
VRQAPLIIPLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLDLKDAFF
CLRLHPTSQPLFAFEWRDPEMGISGQLTWTRLPQGFKNSPTLFNEALHRDLADFRI
QHPDLILLQYVDDLLLAATSELDCQQGTRALLQTLGNLGYRASAKKAQICQKQVKYL
GYLLKEGQRWLTEARKETVMGQPTPKTPRQLREFLGKAGFCRLFIPGFAEMAAPL
YPLTKPGTLFNWGPDQQKAYQEIKQALLTAPALGLPDLTKPFELFVDEKQGYAKGV
LTQKLGPWRRPVAYLSKKLDPVAAGWPPCLRMVAAIAVLTKDAGKLTMGQPLVILA
PHAVEALVKQPPDRWLSNARMTHYQALLLDTDRVQFGPVVALNPATLLPLPEEGL
QHNCLDILAEAHGTRPDLTDQPLPDADHTWYTDGSSLLQEGQRKAGAAVTTETEVI
WAKALPAGTSAQRAELIALTQALKMAEGKKLNVYTDSRYAFATAHIHGEIYRRRGW
LTSEGKEIKNKDEILALLKALFLPKRLSIIHCPGHQKGHSAEARGNRMADQAARKAAI
TETPDTSTLLIENSSPSGGSKRTADGSEFEPKKKRKV
SEQ ID NO: 13 N-terminus fragment of Split 1054
MDYKDHDGDYKDHDIDYKDDDDKMAPKKKRKVGRGMDKKYSIGLAIGTNS
VGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLS
DILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQN
GRDMYVDQELDINRLSDYDVDAIVPQSFLKDDSIDNKVLTRSDKNRGKSDNVPSEE
VVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFF
KTEITLANGCLAGDTLITLADGRRVPIRELVSQQNFSVWALNPQTYRLERARVSRAF
CTGIKPVYRLTTRLGRSIRATANHRFLTPQGWKRVDELQPGDYLALPRRIPTAS
SEQ ID NO: 14 C-terminus fragment of Split 1054
MKRTADGSEFESPRKKRKVDKKYSIGLDIGTNSVGWAVITDEYKVPSKKFKV
LGNTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYD
VDAIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLI
REVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGSGGSSGGSS
GSETPGTSESATPESSGGSSGGSSCLSYETEILTVEYGLLPIGKIVEKRIECTVYSVD
NNGNIYTQPVAQWHDRGEQEVFEYCLEDGSLIRATKDHKFMTVDGQMLPIDEIFER
ELDLMRVDNLPNSGGSPKKKRKVPKKKRKV
SEQ ID NO: 15 N-terminus fragment of Split 1115
MKRTADGSEFESPRKKRKVDKKYSIGLDIGTNSVGWAVITDEYKVPSKKFKV
LGNTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYD
VDAIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLI
REVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNSGGGGS
GGGGSGGGGSCLAGDTLITLADGRRVPIRELVSQQNFSVWALNPQTYRLERARVS
RAFCTGIKPVYRLTTRLGRSIRATANHRFLTPQGWKRVDELQPGDYLALPRRIPTAS
SEQ ID NO: 16 C-terminus fragment of Split 1115
MAAACPELRQLAQSDVYWDPIVSIEPDGVEEVFDLTVPGPHNFVANDIIAHN
GGGGSGGGGSGGGGSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKHYLDEIIE
QISEFSKRVILADANLDKVLSAYNKHRDKPIREQAENIIHLFTLTNLGAPAAFKYFDTT
IDRKRYTSTKEVLDATLIHQSITGLYETRIDLSQLGGDSGGSSGGSSGSETPGTSES
ATPESSGGSSGGSSTLNIEDEYRLHETSKEPDVSLGSTWLSDFPQAWAETGGMGL
AVRQAPLIIPLKATSTPVSIKQYPMSQEARLGIKPHIQRLLDQGILVPCQSPWNTPLL
PVKKPGTNDYRPVQDLREVNKRVEDIHPTVPNPYNLLSGLPPSHQWYTVLDLKDAF
FCLRLHPTSQPLFAFEWRDPEMGISGQLTWTRLPQGFKNSPTLFNEALHRDLADF
RIQHPDLILLQYVDDLLLAATSELDCQQGTRALLQTLGNLGYRASAKKAQICQKQVK
YLGYLLKEGQRWLTEARKETVMGQPTPKTPRQLREFLGKAGFCRLFIPGFAEMAA
PLYPL
SEQ ID NO: 17 pegRNA
CGATTTCTTGGCTTTATATATCTTGTGGAAAGGACGAAACACCGGCAACT
ACAAGACCCGCGCCGGTTTTAGAGCTAGAAATAGCAAGTTAAAATAAGGCTAGT
CCGTTATCAACTTGAAAAAGTGGCACCGAGTCGGTGCCCTCGAACTICACCTCG
GCGCGGGTCTTTTTTTT
SEQ ID NO: 18 DBL pegRNA 1
CGATTTCTTGGCTTTATATATCTTGTGGAAAGGACGAAACACCGGCAACT
ACAAGACCCGCGCCGGTTTTAGAGCTAGAAATAGCAAGTTAAAATAAGGCTAGT
CCGTTATCAACTTGAAAAAGTGGCACCGAGTCGGTGCCCTCGAACTICACCTCG
GCGCGGGTCTGTTTTAGAGCTAGAAATAGCAAGTTAAAATAAGGCTAGTCCGTT
ATCAACTTGAAAAAGTGGCACCGAGTCGGTGCTTTTTTT
SEQ ID NO: 19 DBL pegRNA 2
CGATTTCTTGGCTTTATATATCTTGTGGAAAGGACGAAACACCGGCAACT
ACAAGACCCGCGCCGGTTTTAGAGCTAGAAATAGCAAGTTAAAATAAGGCTAGT
CCGTTATCAACTTGAAAAAGTGGCACCGAGTCGGTGCCCTCGAACTICACCTCG
GCGCGGGTCTTTTTTAGAGCTAGAAATAGCAAGTTAAAATAAGGCTAGTCCGTT
ATCAACTTGAAAAAGTGGCACCGAGTCGGTGCTTTTTTT
SEQ ID NO: 20 DBL pegRNA 3
CGATTTCTTGGCTTTATATATCTTGTGGAAAGGACGAAACACCGGCAACT
ACAAGACCCGCGCCGGTTTTAGAGCTAGAAATAGCAAGTTAAAATAAGGCTAGT
CCGTTATCAACTTGAAAAAGTGGCACCGAGTCGGTCCTCGAACTICACCTCGGC
GCGGGTCTGTTTTAGAGCTAGAAATAGCAAGTTAAAATAAGGCTAGTCCGTTAT
CAACTTGAAAAAGTGGCACCGAGTCGGTGCTTTTTTT
SEQ ID NO: 21 DBL pegRNA 4
CGATTTCTTGGCTTTATATATCTTGTGGAAAGGACGAAACACCGGCAACT
ACAAGACCCGCGCCGGTTTTAGAGCTAGAAATAGCAAGTTAAAATAAGGCTAGT
CCGTTATCAACTTGAAAAAGTGGCACCGAGTCGGTCCTCGAACTICACCTCGGC
GCGGGTCTTTTTTAGAGCTAGAAATAGCAAGTTAAAATAAGGCTAGTCCGTTAT
CAACTTGAAAAAGTGGCACCGAGTCGGTGCTTTTTTT
SEQ ID NO: 22 linker
SGGSSGGSSGSETPGTSESATPESSGGSSGGSST
SEQ ID NO: 23 linker
SGGSSGGSSGSETPGTSESATPESSGGSSGGSSTLEPGEKPYKCPECGKS
FSQSGALTRHQRTHTRDKKYSIGLDIGTNSVGWAVITDEYKVPLE
SEQ ID NO: 24 linker
ASGGGGSGGGGSGGGGSGGGGSGGGGSLE
SEQ ID NO: 25 linker
SGGSSGGSSGSETPGTSESATPESSGGSSGGSST
SEQ ID NO: 26 linker
ASASSGGSSGGSSGSETPGTSESATPESSGGSSGGSGGGGSGGGGSGG
GGSGGGGSGGGGSGTLE

What is claimed is:

1. A prime editor comprising:
(a) a first polynucleotide molecule encoding a Cas protein and an N-terminal fragment of a dimerization protein; and
(b) a second polynucleotide molecule encoding a C-terminal fragment of a dimerization protein and a reverse transcriptase.

2. The prime editor of claim 1, wherein the N-terminal fragment of a dimerization protein is N-terminal fragment of an intein.

3. The prime editor of claim 1, wherein the C-terminal fragment of a dimerization protein is C-terminal fragment of an intein.

4. The prime editor of claim 1, wherein the Cas protein is a Cas nickase, a Cas9 nickase, a dead Cas protein, a dead Cas9, or an active Cas protein.

5. The prime editor of claim 1, wherein the first polynucleotide molecule and the second polynucleotide molecule each comprise a promoter.

6. The prime editor of claim 4, wherein the Cas9 nickase is a Cas9 protein having an amino acid substitution at position 10, at position 840, or at position 863.

7. The prime editor of claim 6, wherein the Cas9 nickase is D1 OA Cas9, D1 ON Cas9, H840N Cas9, H840Y Cas9, H840A Cas9, or N863A Cas9.

8. The prime editor of claim 1, wherein the C-terminal fragment of a dimerization protein and the N-terminal fragment of a dimerization protein are derived from PhoRadA, RmaDnaB^{Δ286}, SspDnaB^{Δ275}, SspDnaB^{M86Δ275}, SspDnaX, TvoVMA, NpuDnaE, NpuDnaB^{Δ283}, SspGyrB, AceL-TerL, PchPRP8, PfuRIR1-1, Psp-GDBPol-1, MtuRecA^{Δ228}, PfuRIR1-2, SceVMA^{Δ206}, RmaDnaB^{Δ271}, MtuRecA^{Δ285}, SspDnaB^{Δ274}, gp41-8, SceVMA^{Δ227}, IMPDH-1, NrdJ-1, MtuRecA^{Δ297}, gp41-1, AovDnaE, AspDnaE, AvaDnaE, Cra(C5505)DnaE, Csp(CCY0110)DnaE, Csp(PCC8801)DnaE, CwaDnaE, Maer(NIES843)DnaE, Mcht(PCC7420)DnaE, MtuRecA^{Δ300}, NspDnaE, OliDnaE, Sel(PC7942)DnaE, SspDnaE, Ssp(PCC7002)DnaE, TerDnaE-3, TelDnaE, TvuDnaE, NeqPol, TerThyX^{Δ132}, or combinations thereof.

9. The prime editor of claim 2, wherein an amino acid sequence of the N-terminal fragment of an intein comprises SEQ ID NO:3.

10. The prime editor of claim 3, wherein an amino acid sequence of the C-terminal fragment of an intein comprises SEQ ID NO:4.

11. The prime editor of claim 1, wherein the reverse transcriptase is an M-MLV reverse transcriptase, a Marathon reverse transcriptase, a Rous sarcoma virus reverse transcriptase, an HIV-1 reverse transcriptase, an AMV reverse transcriptase, a telomerase reverse transcriptase, or any variant thereof.

12. The prime editor of claim 1, wherein the first polynucleotide molecule comprises one or more nuclear localization signals and wherein the second polynucleotide molecule comprises one or more nuclear localization signals.

13. The prime editor of claim 1, wherein the first polynucleotide encodes a polypeptide molecule comprising SEQ ID NO:1.

14. The prime editor of claim 1, wherein the second polynucleotide molecule further comprises a linker.

15. The prime editor of claim 14, wherein the linker encodes a polypeptide molecule comprising SEQ ID NO:22, 23, 24, 25, or 26.

16. The prime editor of claim 1, wherein the first polynucleotide molecule comprises one or more polynucleotides encoding a protein tag and wherein the second polynucleotide molecule comprises one or more polynucleotides encoding a protein tag.

17. A system for prime editing comprising:
(a) a first vector comprising a first polynucleotide molecule encoding a Cas protein and an N-terminal fragment of a dimerization protein; and
(b) a second vector comprising a second polynucleotide molecule encoding a C-terminal fragment of a dimerization protein and a reverse transcriptase.

18. The system of claim 17, wherein the Cas protein is a Cas nickase, Cas9 nickase, a dead Cas protein, a dead Cas9, or an active Cas protein.

19. A prime editor comprising:
(a) a first polynucleotide molecule encoding a reverse transcriptase and an N-terminal fragment of a dimerization protein; and
(b) a second polynucleotide molecule encoding a C-terminal fragment of a dimerization protein and a Cas protein.

20. The prime editor of claim 19, wherein the N-terminal fragment of a dimerization protein is N-terminal fragment of an intein.

21. The prime editor of claim 19, wherein the C-terminal fragment of a dimerization protein is C-terminal fragment of an intein.

22. The prime editor of claim 19, wherein the Cas protein is a Cas nickase, a Cas9 nickase, a dead Cas protein, a dead Cas9, or an active Cas protein.

23. The prime editor of claim 22, wherein the Cas9 nickase is a Cas9 protein having an amino acid substitution at position 10, at position 840, or at position 863.

24. The prime editor of claim 22, wherein the Cas9 nickase is D10A Cas9, D10N Cas9, H840N Cas9, H840Y Cas9, H840A Cas9, or N863A Cas9.

25. The prime editor of claim 19, wherein the first polynucleotide molecule and the second polynucleotide molecule each comprise a promoter.

26. The prime editor of claim 19, wherein the C-terminal fragment of a dimerization protein and the N-terminal fragment of a dimerization protein are derived from PhoRadA, RmaDnaB^{Δ286}, SspDnaB^{Δ275}, SspDnaB^{M86Δ275}, SspDnaX, TvoVMA, NpuDnaE, NpuDnaB^{Δ283}, SspGyrB, AceL-TerL, PchPRP8, PfuRIR1-1, Psp-GDBPol-1, MtuRecA^{Δ228}, PfuRIR1-2, SceVMA^{Δ206}, RmaDnaB^{Δ271}, MtuRecA^{Δ285} SspDnaB^{Δ274}, gp41-8, SceVMA^{Δ227}, IMPDH-1, NrdJ-1, MtuRecA^{Δ297}, gp41-1, AovDnaE, AspDnaE, AvaDnaE, Cra(C5505)DnaE, Csp(CCY0110)DnaE, Csp(PCC8801)DnaE, CwaDnaE, Maer(NIES843)DnaE, Mcht(PCC7420)DnaE, MtuRecA^{Δ300}, NspDnaE, OliDnaE, Sel(PC7942)DnaE, SspDnaE, Ssp(PCC7002)DnaE, TerDnaE-3, TelDnaE, TvuDnaE, NeqPol, TerThyX^{Δ132}, or combinations thereof.

27. The prime editor of claim 20, wherein an amino acid sequence of the N-terminal fragment of an intein comprises SEQ ID NO:3.

28. The prime editor of claim 21, wherein an amino acid sequence of the C-terminal fragment of an intein comprises SEQ ID NO:4.

29. The prime editor of claim 19, wherein the reverse transcriptase is an M-MLV reverse transcriptase, a Marathon reverse transcriptase, a Rous sarcoma virus reverse transcriptase, an HIV-1 reverse transcriptase, an AMV reverse transcriptase, a telomerase reverse transcriptase, or any variant thereof.

30. The prime editor of claim 19, wherein the first polynucleotide molecule comprises one or more nuclear localization signals and wherein the second polynucleotide molecule comprises one or more nuclear localization signals.

31. The prime editor of claim 19, wherein the first polynucleotide molecule further comprises a linker.

32. The prime editor of claim 31, wherein the linker encodes a polypeptide molecule comprising SEQ ID NO:22, 23, 24, 25, or 26.

33. The prime editor of claim 19, wherein the first polynucleotide molecule comprises one or more polynucleotides encoding a protein tag and wherein the second polynucleotide molecule comprises one or more polynucleotides encoding a protein tag.

34. A system for prime editing comprising:
(a) a first vector comprising a first polynucleotide molecule encoding a reverse transcriptase and an N-terminal fragment of a dimerization protein; and
(b) a second vector comprising a second polynucleotide molecule encoding a C-terminal fragment of a dimerization protein and a Cas protein.

35. The system of claim 34, wherein the Cas protein is a Cas nickase, Cas9 nickase, a dead Cas protein, a dead Cas9, or an active Cas protein.

36. A prime editor comprising:
(a) a first polynucleotide molecule encoding a reverse transcriptase, an N-terminal fragment of Cas protein, and an N-terminal fragment of a dimerization protein; and
(b) a second polynucleotide molecule encoding a C-terminal fragment of a dimerization protein and a C-terminal fragment of Cas protein,
wherein the N-terminal fragment of the Cas protein and the C-terminal fragment of the Cas protein when combined form a full-length Cas protein.

37. The prime editor of claim 36, wherein the N-terminal fragment of a dimerization protein is N-terminal fragment of an intein.

38. The prime editor of claim 36, wherein the C-terminal fragment of a dimerization protein is C-terminal fragment of an intein.

39. The prime editor of claim 36, wherein the Cas protein is a Cas nickase, a Cas9 nickase, a dead Cas protein, a dead Cas9, or an active Cas protein.

40. The prime editor of claim 39, wherein the Cas9 nickase is split into a N-terminal fragment and a C-terminal fragment at a split point.

41. The prime editor of claim 40, wherein:
(a) the split point is localized at any amino acid between position 564 and 584, and the N-terminal fragment of Cas9 nickase comprises nucleotides from position 1 of the Cas9 nickase to the split point and the C-terminal fragment of Cas9 nickase comprises nucleotides from the split point to position 1371 of the Cas9 nickase;
(b) the split point is localized at any amino acid between position 249 and 269, and the N-terminal fragment of Cas9 nickase comprises nucleotides from position 1 of the Cas9 nickase to the split point and the C-terminal fragment of Cas9 nickase comprises nucleotides from the split point to position 1371 of the Cas9 nickase; or
(c) the split point is localized at any amino acid between position 265 and 285, and the N-terminal fragment of Cas9 nickase comprises nucleotides from position 1 of the Cas9 nickase to the split point and the C-terminal fragment of Cas9 nickase comprises nucleotides from the split point to position 1371 of the Cas9 nickase.

42. The prime editor of claim 36, wherein the first polynucleotide molecule and the second polynucleotide molecule each comprise a promoter.

43. The prime editor of claim 39, wherein the Cas9 nickase is a Cas9 protein having an amino acid substitution at position 10, at position 840, or at position 863.

44. The prime editor of claim 43, wherein the Cas9 nickase is D10A Cas9, D10N Cas9, H840N Cas9, H840Y Cas9, H840A Cas9, or N863A Cas9.

45. The prime editor of claim 36, wherein the C-terminal fragment of a dimerization protein and the N-terminal fragment of a dimerization protein are derived from PhoRadA, RmaDnaB^{Δ286}, SspDnaB^{Δ275}, SspDnaB^{M86Δ275}, SspDnaX, TvoVMA, NpuDnaE, NpuDnaB^{Δ283}, SspGyrB, AceL-TerL, PchPRP8, PfuRIR1-1, Psp-GDBPol-1, MtuRecA^{Δ228}, PfuRIR1-2, SceVMA^{Δ206}, RmaDnaB^{Δ271}, MtuRecA^{Δ285} SspDnaB^{Δ274}, gp41-8, SceVMA^{Δ227}, IMPDH-1, NrdJ-1, MtuRecA^{Δ297}, gp41-1, AovDnaE, AspDnaE, AvaDnaE, Cra(C5505)DnaE, Csp(CCY0110)DnaE, Csp(PCC8801)DnaE, CwaDnaE, Maer(NIES843)DnaE, Mcht(PCC7420)DnaE, MtuRecA^{Δ300}, NspDnaE, OliDnaE, Sel(PC7942)DnaE, SspDnaE, Ssp(PCC7002)DnaE, TerDnaE-3, TelDnaE, TvuDnaE, NeqPol, TerThyX^{Δ132}, or combinations thereof.

46. The prime editor of claim 36, wherein the reverse transcriptase is an M-MLV reverse transcriptase, Marathon reverse transcriptase, a Rous sarcoma virus reverse transcriptase, an HIV-1 reverse transcriptase, an AMV reverse transcriptase, a telomerase reverse transcriptase, or any variant thereof.

47. The prime editor of claim 36, wherein the first polynucleotide molecule comprises one or more nuclear localization signals and wherein the second polynucleotide molecule comprises one or more nuclear localization signals.

48. The prime editor of claim 36, wherein a sequence of the first polynucleotide molecule encodes a polypeptide comprising SEQ ID NO:5.

49. The prime editor of claim 37, wherein an amino acid sequence of the N-terminal fragment of an intein comprises SEQ ID NO:3.

50. The prime editor of claim 36, wherein a sequence of the second polynucleotide molecule encodes a polypeptide comprising SEQ ID NO:6.

51. The prime editor of claim 38, wherein an amino acid sequence of the C-terminal fragment of an intein comprises SEQ ID NO:7.

52. The prime editor of claim 36, wherein the first polynucleotide molecule further comprises a linker.

53. The prime editor of claim 52, wherein the linker encodes a polypeptide molecule comprising SEQ ID NO:22, 23, 24, 25, or 26.

54. The prime editor of claim 36, wherein the first polynucleotide molecule comprises one or more polynucleotides encoding a protein tag and wherein the second polynucleotide molecule comprises one or more polynucleotides encoding a protein tag.

55. A system for prime editing comprising:
(a) a first vector comprising a first polynucleotide molecule encoding a reverse transcriptase, an N-terminal fragment of Cas protein, and an N-terminal fragment of a dimerization protein;
(b) a second vector comprising a second polynucleotide molecule encoding a C-terminal fragment of a dimerization protein and a C-terminal fragment of Cas protein,
wherein the N-terminal fragment of the Cas protein and the C-terminal fragment of the Cas protein when combined form a full-length Cas protein.

56. The system of claim 55, wherein the N-terminal fragment of a dimerization protein is N-terminal fragment of an intein.

57. The system of claim 55, wherein the C-terminal fragment of a dimerization protein is C-terminal fragment of an intein.

58. The system of claim 55, wherein the Cas protein is a Cas nickase, a Cas9 nickase, a dead Cas protein, a dead Cas9, or an active Cas protein.

59. A prime editor comprising:
(a) a first polynucleotide molecule encoding a N-terminal fragment of a Cas protein, and a N-terminal fragment of a dimerization protein; and
(b) a second polynucleotide molecule encoding a C-terminal fragment of a dimerization protein, a C-terminal fragment of Cas protein, and a reverse transcriptase,
wherein the N-terminal fragment of the Cas protein and the C-terminal fragment of the Cas protein when combined form a full-length Cas protein.

60. The prime editor of claim 59, wherein the N-terminal fragment of a dimerization protein is N-terminal fragment of an intein.

61. The prime editor of claim 59, wherein the C-terminal fragment of a dimerization protein is C-terminal fragment of an intein.

62. The prime editor of claim 59, wherein the Cas protein is a Cas nickase, a Cas9 nickase, a dead Cas protein, a dead Cas9, or an active Cas protein.

63. The prime editor of claim 62, wherein the Cas9 nickase is split into a N-terminal fragment and a C-terminal fragment at a split point.

64. The prime editor of claim 63, wherein:
(a) the split point is localized at any amino acid between position 703 and 723, and the N-terminal fragment of Cas9 nickase comprises nucleotides from position 1 of the Cas9 nickase to the split point and the C-terminal fragment of Cas9 nickase comprises nucleotides from the split point to position 1371 of the Cas9 nickase;
(b) the split point is localized at any amino acid between position 935 and 965, and the N-terminal fragment of Cas9 nickase comprises nucleotides from position 1 of the Cas9 nickase to the split point and the C-terminal fragment of Cas9 nickase comprises nucleotides from the split point to position 1371 of the Cas9 nickase;
(c) the split point is localized at any amino acid between position 1044 and 1064 and, the N-terminal fragment of Cas9 nickase comprises nucleotides from position 1 of the Cas9 nickase to the split point and the C-terminal fragment of Cas9 nickase comprises nucleotides from the split point to position 1371 of the Cas9 nickase; or
(d) the split point is localized at any amino acid between position 1105 and 1125, and the N-terminal fragment of Cas9 nickase comprises nucleotides from position 1 of the Cas9 nickase to the split point and the C-terminal fragment of Cas9 nickase comprises nucleotides from the split point to position 1371 of the Cas9 nickase.

65. The prime editor of claim 59, wherein the first polynucleotide molecule and the second polynucleotide molecule each comprise a promoter.

66. The prime editor of claim 62, wherein the Cas9 nickase is a Cas9 protein having an amino acid substitution at position 10, at position 840, or at position 863.

67. The prime editor of claim 66, wherein the Cas9 nickase is D10A Cas9, D10N Cas9, H840N Cas9, H840Y Cas9, H840A Cas9, or N863A Cas9.

68. The prime editor of claim 59, wherein the C-terminal fragment of a dimerization protein and the N-terminal fragment of a dimerization protein are derived from PhoRadA, RmaDnaB^{Δ286}, SspDnaB^{Δ275}, SspDnaB^{M86Δ275}, SspDnaX, TvoVMA, NpuDnaE, NpuDnaB^{Δ283}, SspGyrB, AceL-TerL, PchPRP8, PfuRIR1-1, Psp-GDBPol-1, MtuRecA^{Δ228}, PfuRIR1-2, SceVMA^{Δ206}, RmaDnaB^{Δ271}, MtuRecA^{Δ285} SspDnaB^{Δ274}, gp41-8, SceVMA^{Δ227}, IMPDH-1, NrdJ-1, MtuRecA^{Δ297}, gp41-1, AovDnaE, AspDnaE, AvaDnaE, Cra(C5505)DnaE, Csp(CCY0110)DnaE, Csp(PCC8801)DnaE, CwaDnaE, Maer(NIES843)DnaE, Mcht(PCC7420)DnaE, MtuRecA^{Δ300}, NspDnaE, OliDnaE, Sel(PC7942)DnaE, SspDnaE, Ssp(PCC7002)DnaE, TerDnaE-3, TelDnaE, TvuDnaE, NeqPol, TerThyXΔ¹³², or combinations thereof.

69. The prime editor of claim 59, wherein the reverse transcriptase is an M-MLV reverse transcriptase, Marathon reverse transcriptase, a Rous sarcoma virus reverse transcriptase, an HIV-1 reverse transcriptase, an AMV reverse transcriptase, a telomerase reverse transcriptase, or any variant thereof.

70. The prime editor of claim 59, wherein the first polynucleotide molecule comprises one or more nuclear localization signals and wherein the second polynucleotide molecule comprises one or more nuclear localization signals.

71. The prime editor of claim 59, wherein a sequence of the first polynucleotide molecule encodes a polypeptide comprising SEQ ID NO:9, 11, 13, or 15.

72. The prime editor of claim 60, wherein an amino acid sequence of the N-terminal fragment of an intein comprises SEQ ID NO:3.

73. The prime editor of claim 59, wherein a sequence of the second polynucleotide molecule encodes a polypeptide comprising SEQ ID NO:10, 12, 14, or 16.

74. The prime editor of claim 61, wherein an amino acid sequence of the C-terminal fragment of an intein comprises SEQ ID NO:7.

75. The prime editor of claim 59, wherein the first polynucleotide molecule further comprises a linker.

76. The prime editor of claim 75, wherein the linker encodes a polypeptide molecule comprising SEQ ID NO:22, 23, 24, 25, or 26.

77. The prime editor of claim 59, wherein the first polynucleotide molecule comprises one or more polynucleotides encoding a protein tag and wherein the second polynucleotide molecule comprises one or more polynucleotides encoding a protein tag.

78. A system for prime editing comprising:
(a) a first vector comprising a first polynucleotide molecule encoding a N-terminal fragment of a Cas protein, and a N-terminal fragment of a dimerization protein; and
(b) a second vector comprising a second polynucleotide molecule encoding a C-terminal fragment of a dimerization protein, a C-terminal fragment of Cas protein, and a reverse transcriptase,
wherein the N-terminal fragment of the Cas protein and the C-terminal fragment of the Cas protein when combined form a full-length Cas protein.

79. The prime editor of claim 78, wherein the N-terminal fragment of a dimerization protein is N-terminal fragment of an intein.

80. The prime editor of claim 78, wherein the C-terminal fragment of a dimerization protein is C-terminal fragment of an intein.

81. The prime editor of claim 78, wherein the Cas protein is a Cas nickase, a Cas9 nickase, a dead Cas protein, a dead Cas9, or an active Cas protein.

82. A method of editing genomic DNA in a cell comprising contacting the cell with the system for prime editing of claim 17, 34, 55, or 78, wherein the system further comprises one or more pegRNA molecules, one or more sgRNA molecules, or a combination of one or more pegRNA molecules and one or more sgRNA molecules.

83. The method of claim 82, wherein the one or more pegRNA molecule comprise one or more loops, one or more base modifications, or a combination of one or more loops and one or more base modifications to enhance prime editing activity.

84. The method of claim 82, wherein editing genomic DNA does not generate double-stranded break.

85. The method of claim 82, wherein editing genomic DNA induces an insertion, deletion, transversion point mutation, or transition point mutation.

86. The method of claim 82, wherein the first vector and the second vector are AAV vectors.

87. The method of claim 82, wherein the one or more pegRNA molecules comprise SEQ ID NOs:17, 18, 19, 20, or 21.


SEQUENCE LISTING (43 sequences)

SEQ ID NO: 1 - protein, 1515 aa, Artificial Sequence
  Amino acid sequence of the N-terminal fragment of the split prime editor at the linker
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNSGGSKRTADGSEFEPKKKRKV

SEQ ID NO: 2 - protein, 783 aa, Artificial Sequence
  Amino acid sequence of the C-terminus fragment of the split prime editor at the linker
MKRTADGSEFESPKKKRKVIKIATRKYLGKQNVYDIGVERDHNFALKNGFIASNGRAGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 3 - protein, 102 aa, Artificial Sequence
  sequence of the N-terminal fragment of a split intein (Part of SEQ ID NO1)
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 4 - protein, 66 aa, Artificial Sequence
  (Part of SEQ ID NO2)
IKIATRKYLGKQNVYDIGVERDHNFALKNGFIASNGRAGGSSGSETPGTSESATPESSGGSSGGSS

SEQ ID NO: 5 - protein, 1451 aa, Artificial Sequence
  Amino acid sequence of the N-terminus fragment of the Split prime editor with the MMLV at the N-terminus
MGPKKKRKVGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKVSGGSSGGSSGSETPGTSESATPESSGGSSGGSSTLEPGEKPYKCPECGKSFSQSGALTRHQRTHTRDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 6 - protein, 975 aa, Artificial Sequence
  Amino acid sequence of the C-terminus fragment of the Split prime editor with the MMLV at the N-terminus
MKRTADGSEFESPKKKRKVIKIATRKYLGKQNVYDIGVERDHNFALKNGFIASNCFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSYPYDVPDYAYPYDVPDYAYPYDVPDYASGGSPKKKRKV

SEQ ID NO: 7 - protein, 35 aa, Artificial Sequence
  C-terminal fragment of a split intein (Part of SEQ ID NO6)
IKIATRKYLGKQNVYDIGVERDHNFALKNGFIASN

SEQ ID NO: 8 - protein, 426 aa, Artificial Sequence
  Marathon Reverse Transcriptase
NLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTCEFE

SEQ ID NO: 9 - protein, 833 aa, Artificial Sequence
  N-terminus fragment of Split 713
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 10 - protein, 1438 aa, Artificial Sequence
  10 C-terminus fragment of Split 713
MAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 11 - protein, 1060 aa, Artificial Sequence
  11 N-terminus fragment of Split 945
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 12 - protein, 1224 aa, Artificial Sequence
  C-terminus fragment of Split 945
MAAACPELRQLAQSDVYWDPIVSIEPDGVEEVFDLTVPGPHNFVANDIIAHNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 13 - protein, 1192 aa, Artificial Sequence
  N-terminus fragment of Split 1054
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 14 - protein, 1226 aa, Artificial Sequence
  C-terminus fragment of Split 1054
MKRTADGSEFESPRKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGSGGSSGGSSGSETPGTSESATPESSGGSSGGSSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNSGGSPKKKRKVPKKKRKV

SEQ ID NO: 15 - protein, 1251 aa, Artificial Sequence
  15 N-terminus fragment of Split 1115
MKRTADGSEFESPRKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSGGGGSGGGGSGGGGSCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 16 - protein, 679 aa, Artificial Sequence
  C-terminus fragment of Split 1115
MAAACPELRQLAQSDVYWDPIVSIEPDGVEEVFDLTVPGPHNFVANDIIAHNGGGGSGGGGSGGGGSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPL

SEQ ID NO: 17 - DNA, 175 bp, Artificial Sequence
  pegRNA
cgatttcttggctttatatatcttgtggaaaggacgaaacaccggcaactacaagacccgcgccggttttagagctagaaatagcaagttaaaataaggctagtccgttatcaacttgaaaaagtggcaccgagtcggtgccctcgaacttcacctcggcgcgggtctttttttt

SEQ ID NO: 18 - DNA, 251 bp, Artificial Sequence
  DBL pegRNA 1
cgatttcttggctttatatatcttgtggaaaggacgaaacaccggcaactacaagacccgcgccggttttagagctagaaatagcaagttaaaataaggctagtccgttatcaacttgaaaaagtggcaccgagtcggtgccctcgaacttcacctcggcgcgggtctgttttagagctagaaatagcaagttaaaataaggctagtccgttatcaacttgaaaaagtggcaccgagtcggtgcttttttt

SEQ ID NO: 19 - DNA, 251 bp, Artificial Sequence
  DBL pegRNA 2
cgatttcttggctttatatatcttgtggaaaggacgaaacaccggcaactacaagacccgcgccggttttagagctagaaatagcaagttaaaataaggctagtccgttatcaacttgaaaaagtggcaccgagtcggtgccctcgaacttcacctcggcgcgggtcttttttagagctagaaatagcaagttaaaataaggctagtccgttatcaacttgaaaaagtggcaccgagtcggtgcttttttt

SEQ ID NO: 20 - DNA, 249 bp, Artificial Sequence
  DBL pegRNA 3
cgatttcttggctttatatatcttgtggaaaggacgaaacaccggcaactacaagacccgcgccggttttagagctagaaatagcaagttaaaataaggctagtccgttatcaacttgaaaaagtggcaccgagtcggtcctcgaacttcacctcggcgcgggtctgttttagagctagaaatagcaagttaaaataaggctagtccgttatcaacttgaaaaagtggcaccgagtcggtgcttttttt

SEQ ID NO: 21 - DNA, 249 bp, Artificial Sequence
  DBL pegRNA 4
cgatttcttggctttatatatcttgtggaaaggacgaaacaccggcaactacaagacccgcgccggttttagagctagaaatagcaagttaaaataaggctagtccgttatcaacttgaaaaagtggcaccgagtcggtcctcgaacttcacctcggcgcgggtcttttttagagctagaaatagcaagttaaaataaggctagtccgttatcaacttgaaaaagtggcaccgagtcggtgcttttttt

SEQ ID NO: 22 - protein, 34 aa, Artificial Sequence
  linker
SGGSSGGSSGSETPGTSESATPESSGGSSGGSST

SEQ ID NO: 23 - protein, 95 aa, Artificial Sequence
  Linker
SGGSSGGSSGSETPGTSESATPESSGGSSGGSSTLEPGEKPYKCPECGKSFSQSGALTRHQRTHTRDKKYSIGLDIGTNSVGWAVITDEYKVPLE

SEQ ID NO: 24 - protein, 29 aa, Artificial Sequence
  Linker
ASGGGGSGGGGSGGGGSGGGGSGGGGSLE

SEQ ID NO: 25 - protein, 34 aa, Artificial Sequence; identical to SEQ ID NO: 22
  Linker

SEQ ID NO: 26 - protein, 65 aa, Artificial Sequence
  Linker
ASASSGGSSGGSSGSETPGTSESATPESSGGSSGGSGGGGSGGGGSGGGGSGGGGSGGGGSGTLE

SEQ ID NO: 27 - protein, 7 aa, Artificial Sequence
  monopartite NLS from the SV40 Large T-antigen
PKKKRKV

SEQ ID NO: 28 - protein, 16 aa, Artificial Sequence
  NLS of nucleoplasmin, KR[PAATKKAGQA]KKKK is an example of bipartite signal
KRPAATKKAGQAKKKK

SEQ ID NO: 29 - DNA, 21 bp, Artificial Sequence
  No prime editor
gccgaggtgtagttcgagggc

SEQ ID NO: 30 - DNA, 21 bp, Artificial Sequence
  No prime editor
cggctccacatcaagctcccg

SEQ ID NO: 31 - DNA, 21 bp, Artificial Sequence
  Prime editor
gccgaggtgaagttcgagggc

SEQ ID NO: 32 - DNA, 21 bp, Artificial Sequence
  Prime editor
cggctccacttcaagctcccg

SEQ ID NO: 33 - DNA, 47 bp, Artificial Sequence
  Portion of PE2
aagggcctgagtccgagcagaagaagaagggctcccatcacatcaac

SEQ ID NO: 34 - DNA, 47 bp, Artificial Sequence
  Portion of PE2
  misc_feature (30)..(30): n is a, c, g, t or u
aagggcctgagtccgagcagaagaagaagngctcccatcacatcaac

SEQ ID NO: 35 - protein, 7 aa, Artificial Sequence
  Stop codon removed fluorescent
AEVKFEG

SEQ ID NO: 36 - DNA, 22 bp, Artificial Sequence
  Portion of PE2
gagcagaagaagaagggctccc

SEQ ID NO: 37 - DNA, 19 bp, Artificial Sequence
  Portion of PE2 (N-Terminus MMLV)
cagaagaagaagggctccc

SEQ ID NO: 38 - DNA, 56 bp, Artificial Sequence
  Portion of split N-Terminus MMLV PE2
gaagggcctgagtccgagcagaagaagaagggctcccatcacatcaaccggtggcg

SEQ ID NO: 39 - DNA, 175 bp, Artificial Sequence
  pegRNA
gctaaagaaccgaaatatatagaacacctttcctgctttgtggccgttgatgttctgggcgcggccaaaatctcgatctttatcgttcaattttattccgatcaggcaatagttgaactttttcaccgtggctcagccacgggagcttgaagtggagccgcgcccagaaaaaaaa

SEQ ID NO: 40 - DNA, 251 bp, Artificial Sequence
  DBL pegRNA 1
gctaaagaaccgaaatatatagaacacctttcctgctttgtggccgttgatgttctgggcgcggccaaaatctcgatctttatcgttcaattttattccgatcaggcaatagttgaactttttcaccgtggctcagccacgggagcttgaagtggagccgcgcccagacaaaatctcgatctttatcgttcaattttattccgatcaggcaatagttgaactttttcaccgtggctcagccacgaaaaaaa

SEQ ID NO: 41 - DNA, 251 bp, Artificial Sequence
  DBL pegRNA 2
gctaaagaaccgaaatatatagaacacctttcctgctttgtggccgttgatgttctgggcgcggccaaaatctcgatctttatcgttcaattttattccgatcaggcaatagttgaactttttcaccgtggctcagccacgggagcttgaagtggagccgcgcccagaaaaaatctcgatctttatcgttcaattttattccgatcaggcaatagttgaactttttcaccgtggctcagccacgaaaaaaa

SEQ ID NO: 42 - DNA, 249 bp, Artificial Sequence
  DBL pegRNA 3
gctaaagaaccgaaatatatagaacacctttcctgctttgtggccgttgatgttctgggcgcggccaaaatctcgatctttatcgttcaattttattccgatcaggcaatagttgaactttttcaccgtggctcagccaggagcttgaagtggagccgcgcccagacaaaatctcgatctttatcgttcaattttattccgatcaggcaatagttgaactttttcaccgtggctcagccacgaaaaaaa

SEQ ID NO: 43 - DNA, 249 bp, Artificial Sequence
  DBL pegRNA 4
gctaaagaaccgaaatatatagaacacctttcctgctttgtggccgttgatgttctgggcgcggccaaaatctcgatctttatcgttcaattttattccgatcaggcaatagttgaactttttcaccgtggctcagccaggagcttgaagtggagccgcgcccagaaaaaatctcgatctttatcgttcaattttattccgatcaggcaatagttgaactttttcaccgtggctcagccacgaaaaaaa